(19) United States
(12) Patent Application Publication
(10) Pub. No.: US 2023/0367784 A1
(43) Pub. Date: Nov. 16, 2023

(54) SYSTEM FOR AUTOMATED EXTRACTION OF ANALYTICAL INSIGHTS FROM AN INTEGRATED LUNG NODULE PATIENT MANAGEMENT APPLICATION

(71) Applicant: KONINKLIJKE PHILIPS N.V., Eindhoven (NL)

(72) Inventors: IGOR JACOBS, ASTEN (NL); DARSHAN SAKARAYAPATNA, EINDHOVEN (NL); SANKALP SIPAULYA, BANGALORE (IN); ROBERT CHRISTIAAN VAN OMMERING, CAMBRIDGE, MA (US); JOSEPH JAYAKAR NALLURI, CAMBRIDGE, MA (US); ELTON HEDDEN, ANDOVER, MA (US)

(21) Appl. No.: 18/090,787

(22) Filed: Dec. 29, 2022

Related U.S. Application Data
(60) Provisional application No. 63/342,340, filed on May 16, 2022.

(51) Int. Cl. G06F 16/25; G16H 40/20; G06F 16/26

(52) U.S. Cl. CPC G06F 16/254; G16H 40/20; G06F 16/26

(57) ABSTRACT

In one embodiment, a method performed by a computing device executing an analytics application used in conjunction with a patient management application, the method comprising: receiving workflows and events from the patient management application, the workflows and events corresponding to patient data; selectively processing the workflows and events in extract, transform, and load (ETL) pipelines responsive to trigger points in the workflows; and loading, by the ETL pipelines, data resulting from the selective processing into a data analytics data structure used to enable visualization of patient data and derived metrics or key performance indicators.

### CROSS REFERENCE TO RELATED APPLICATIONS

The present application claims priority to U.S. Provisional Patent Application No. 63/342,340 filed May 16, 2022.

### FIELD OF THE INVENTION

The present invention is generally related to patient management systems, and more particularly, analytics for lung cancer patient management applications for patients in lung cancer screening and incidental pulmonary findings programs.

### BACKGROUND OF THE INVENTION

Clinicians and leadership of patient management systems, including lung nodule management programs, do not have effective ways to collect and report out on clinical, operational and financial key performance indicators. This is due to the lack of structured data, the lack of resources to collect the data and the lack of accessibility to data. Analytical insights are often not available at all, or require manual capture and aggregation of data from the hospital's information systems. Not only is such effort very labor intensive, data are also often captured in flat data sheets, without the ability to effectively inspect, or report out, on them. As a consequence, it is difficult for clinicians or program management to track how many screening or incidental exams are being reviewed, what are the next steps and follow-up decisions, and what are the outcomes of the tests in the program. This results in a lack of insight in the clinical outcomes of, for instance, the lung nodule management programs, their operational efficacy (including staffing) and revenue.

### SUMMARY OF THE INVENTION

In one embodiment, a method performed by a computing device executing an analytics application used in conjunction with a patient management application, the method comprising: receiving workflows and events from the patient management application, the workflows and events corresponding to patient data; selectively processing the workflows and events in extract, transform, and load (ETL) pipelines responsive to trigger points in the workflows; and loading, by the ETL pipelines, data resulting from the selective processing into a data analytics data structure used to enable visualization of patient data and derived metrics or key performance indicators.

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic diagram that illustrates an example high level architecture of an analytics application, in accordance with an embodiment of the invention.

FIG. 2 is a schematic diagram that illustrates example workflows in a lung nodule management application, in accordance with an embodiment of the invention.

FIG. 3 is a schematic diagram that illustrates example main states of a lung screening workflow, in accordance with an embodiment of the invention.

FIGS. 4A-4B are schematic diagrams that illustrate example entity tree objects and their creation/updates in a lung screening process, in accordance with an embodiment of the invention.

FIG. 5 is a schematic diagram that illustrates example main states of a lung incidental findings workflow, in accordance with an embodiment of the invention.

FIG. 6 is a schematic diagram that illustrates an example overall design of an analytics application and ETL (extract, transform, load) in a cloud based software as a service system, in accordance with an embodiment of the invention.

FIG. 7 is a schematic diagram that illustrates example relevant entity tree objects for a lung analytics ETL, in accordance with an embodiment of the invention.

FIG. 8 is a schematic diagram that illustrates an example top-level ETL pipeline, in accordance with an embodiment of the invention.

FIG. 9 is a schematic diagram that illustrates an example scheduling strategy of a GenerateFlowFile processor during development, in accordance with an embodiment of the invention.

FIG. 10 is a schematic diagram that illustrates example checks performed by a check arguments processor, in accordance with an embodiment of the invention.

FIG. 11 is a schematic diagram that illustrates finding a last updated time stamp, in accordance with an embodiment of the invention.

FIG. 12 is a schematic diagram that illustrates setting of an Avro to JSON converter, in accordance with an embodiment of the invention.

FIG. 13 is a schematic diagram that illustrates storing last updated information from JSON content into a FlowFile attribute, in accordance with an embodiment of the invention.

FIG. 14 is a schematic diagram that illustrates an example pipeline loop with successful outputs, in accordance with an embodiment of the invention.

FIG. 15 is a schematic diagram that illustrates example error handling in a main pipeline, in accordance with an embodiment of the invention.

FIGS. 16A-16B are schematic diagrams that illustrate an example loop that fetches root objects in chunks, in accordance with an embodiment of the invention.

FIG. 17 is a schematic diagram that illustrates setting a start time to a normalized value of last updated, in accordance with an embodiment of the invention.

FIG. 18 is a schematic diagram that illustrates calculating an end time of a window by adding a window size to a start time, in accordance with an embodiment of the invention.

FIG. 19 is a schematic diagram that illustrates getting a number of entries retrieved from an entity tree, in accordance with an embodiment of the invention.

FIG. 20 is a schematic diagram that illustrates checking a number of entries as retrieved from an entity tree, in accordance with an embodiment of the invention.

FIG. 21 is a schematic diagram that illustrates getting a time stamp of a last retrieved record, in accordance with an embodiment of the invention.

FIG. 22 is a schematic diagram that illustrates calculating a new end time for a current time window if there are more records to be retrieved, in accordance with an embodiment of the invention.

FIG. 23 is a schematic diagram that illustrates splitting an array of records into separate records, in accordance with an embodiment of the invention.

FIG. 24 is a schematic diagram that illustrates determining whether this is the last record of a split, in accordance with an embodiment of the invention.

FIG. 25 is a schematic diagram that illustrates an example process group responsible for performing analytics application specific processing, in accordance with an embodiment of the invention.

FIG. 26 is a schematic diagram that illustrates only triggering a next time fetch if the last record of the previous fetch is being processed, in accordance with an embodiment of the invention.

FIG. 27 is a schematic diagram that illustrates determining whether another fetch is needed, in accordance with an embodiment of the invention.

FIG. 28 is a schematic diagram that illustrates starting a new time window, in accordance with an embodiment of the invention.

FIGS. 29A-29B are schematic diagrams that illustrate example process groups for fetching entity tree objects, in accordance with an embodiment of the invention.

FIG. 30 is a schematic diagram that illustrates an example NiFi design pattern for extracting and transforming information, in accordance with an embodiment of the invention.

FIG. 31 is a schematic diagram that illustrates an example extraction and transformation of patient attributes, in accordance with an embodiment of the invention.

FIG. 32 is a schematic diagram that illustrates putting data into an analytics database, in accordance with an embodiment of the invention.

FIG. 33 is a schematic diagram that illustrates an example of detailed information of each processor inside a process group, in accordance with an embodiment of the invention.

FIGS. 34A-34B are schematic diagrams that illustrate an example lung analytics summary dashboard, in accordance with an embodiment of the invention.

FIGS. 35A-35B are schematic diagrams that illustrate an example lung analytics screening dashboard, in accordance with an embodiment of the invention.

FIGS. 36A-36C are schematic diagrams that illustrate an example lung analytics biopsy and outcomes dashboard, in accordance with an embodiment of the invention.

FIGS. 37A-37C are schematic diagrams that illustrate an example lung analytics clinical outcomes dashboard, in accordance with an embodiment of the invention.

### DETAILED DESCRIPTION OF EMBODIMENTS

Disclosed herein are certain embodiments of an analytics application and associated systems and methods that are implemented in a cloud-based patient health platform. The analytics application is described here in the context of Philips IntelliSpace Precision Medicine (ISPM), which is a cloud-based Software as a Service (SaaS) system hosted on the Philips HealthSuite Digital Platform (HSDP), though it should be appreciated that functionality of the analytics application may be implemented in other platforms in some embodiments, such as the Philips HealthSuite Diagnostics (HSD) platform. In the example embodiments described herein, the analytics application is described in conjunction with (embedded in, or stand-alone and used in conjunction with) the Philips Lung Cancer Orchestrator (LCO), which is an integrated lung cancer patient management system for lung screening and incidental pulmonary findings programs that monitors patients through various steps of their lung cancer detection, diagnosis and treatment decision journey. Again, the examples described below are for illustration, and it should be appreciated that some embodiments of the analytics application may be used in conjunction with other and/or additional lung cancer management systems, other and/or additional applications across the lung care continuum, and/or in cooperation with patient management systems dedicated or involved in patient care for other diseases or health issues.

In one embodiment, the analytics application extracts relevant metrics from workflows captured in LCO via specific NiFi ETL (extract, transform, load) pipelines. The analytics application comprises dedicated pages for screening, incidental findings, biopsy (e.g., tissue and/or liquid) & outcomes and clinical outcomes, displaying insights including: patient volumes, patients per workflow step or follow-up decision, Lung-RADS (screening) or Fleischner (Incidental findings) categories, diagnostic follow-up decisions and breakdown of performed tests, tissue sampling results and lung cancer detection rates. ISPM-integrated intuitive analytics dashboards enable physicians and leadership to comprehend and track the aforementioned metrics in a visual interface within the ISPM platform.

Digressing briefly, an important component for driving lung nodule management programs is having operational and clinical insights in the efficacy and quality of lung nodule management. These insights may be used to monitor lung nodule management programs, report to internal and external stakeholders and drive quality improvement initiatives. As explained above, clinicians and leadership of patient management systems, including lung nodule management programs, do not have effective ways to collect and report out on clinical, operational and financial key performance indicators. Certain embodiments of an analytics application overcome challenges by automated extraction of the relevant datapoints from the patient management software, and deriving key metrics and performance indicators from them through transformation of the data and loading them into integrated intuitive analytics dashboards that enable the physicians and leadership to comprehend and track the aforementioned metrics in a visual interface embedded in the patient management application. These analytical insights play an important role in driving effective and high-quality lung nodule management programs.

Having summarized certain features of an analytics application of the present disclosure, reference will now be made in detail to the description of an analytics application as illustrated in the drawings. While an analytics application will be described in connection with these drawings, there is no intent to limit it to the embodiment or embodiments disclosed herein. For instance, the analytics application may be applicable to various medical domains, including oncology, cardiovascular, etc. That is, the lung analytics application may be configured for other analytics applications (e.g., genome analytics, prostate analytics), or for use with other disease orchestrators (e.g., in addition to or as an alternative to a lung cancer orchestrator, prostate cancer orchestrator, oncology orchestrator, cardiology care orchestrator, neurology orchestrator, etc.). Although described herein for lung cancer screening and incidental pulmonary findings, in some embodiments, the analytics application may be used in conjunction with other incidental findings management applications or other findings management and scheduling and reporting applications. Further, although the description identifies or describes specifics of one or more embodiments, such specifics are not necessarily part of every embodiment, nor are all of any various stated advantages necessarily associated with a single embodiment. The intent is to cover all alternatives, modifications and equivalents included within the principles and scope of the disclosure as defined by the appended claims. As another example, two or more embodiments may be interchanged or combined in any combination. Further, it should be appreciated in the context of the present disclosure that the claims are not necessarily limited to the particular embodiments set out in the description.

Before commencing a description of certain embodiments of an analytics application, it is noted that the description contains references to common NiFi terms, which would be understood to one having ordinary skill in the art. A few examples of these terms are as follows:
Processor: Processors are the basic blocks providing capabilities for data ingestion, transformation, processing, aggregation, etc.
Process Group: A Process Group is a specific set of processes and their connections, which can receive data via input ports and send data out via output ports
Connection: Connections provide the actual linkage between processors
FlowFile: A FlowFile represents each object moving through the system and for each one, NiFi keeps track of a map of key/value pair attribute strings and its associated content of zero or more bytes.

Explaining further, FlowFile is an information package. Each processor has an ability to process the generated FlowFile from a root processor. In the lifecycle of NiFi execution, one file flow across all the processor is named as a FlowFile. Published literature is available for further reading on NiFi, including an Internet article entitled, “Building a Data Pipeline with Apache NiFi”, published by Hadoop in Real World on Jun. 15, 2020. Accordingly, a further general explanation of NiFi and data pipelines is omitted herein except where properties unique to the particulars of the present disclosure are disclosed. Reference to events includes medical exams or other events that may be part of a patient's care journey, from which data are captured. For instance, events may be captured from data fields of the patient management application.

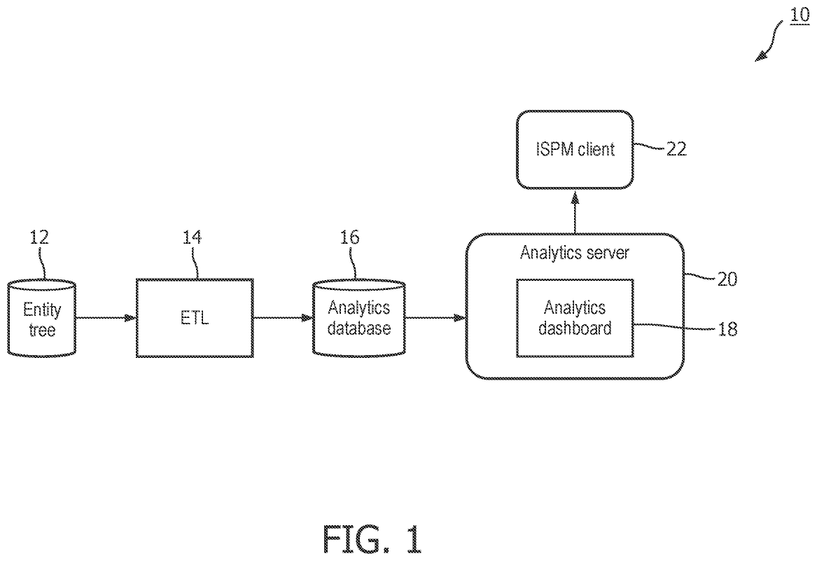

FIG. 1 is a schematic diagram that illustrates an example high level architecture of an analytics application 10, in accordance with an embodiment of the invention. In one embodiment, the analytics application 10 comprises an ISPM entity tree 12, an ETL pipeline 14, an analytics database 16, an analytics server 20 comprising analytics dashboards 18, and an ISPM client 22. Note that in some embodiments, the analytics application 10 comprises fewer (or more) than the functionality depicted in FIG. 1. Briefly, the analytics application 10 comprises a software feature configured in one embodiment as an embedded analytics application on the ISPM platform.

The ISPM entity tree 12 comprises data captured while creating and executing workflows (e.g., actions taken by a user of the patient management application while navigating through patient care steps, including populating fields with patient data in several display user interfaces, ordering, scheduling exams, collecting data, etc.) in ISPM and the results captured while executing these workflows.

The ETL pipeline 14 extracts data from the ISPM entity tree 12, transforms it into a format suitable for analytics, and loads it into the analytics database 16.

The analytics database 16 comprises the data as extracted from the ISPM platform in a format suitable to build the analytics dashboards 18. Note that, although described as a database, other types of data structures may be used in some embodiments.

The analytics dashboards 18 are built on top of the analytics database 16 and provide end-user insights.

The ISPM client 22 makes the analytics dashboards 18 available to an end-user(s) via embedded analytics pages in the ISPM.

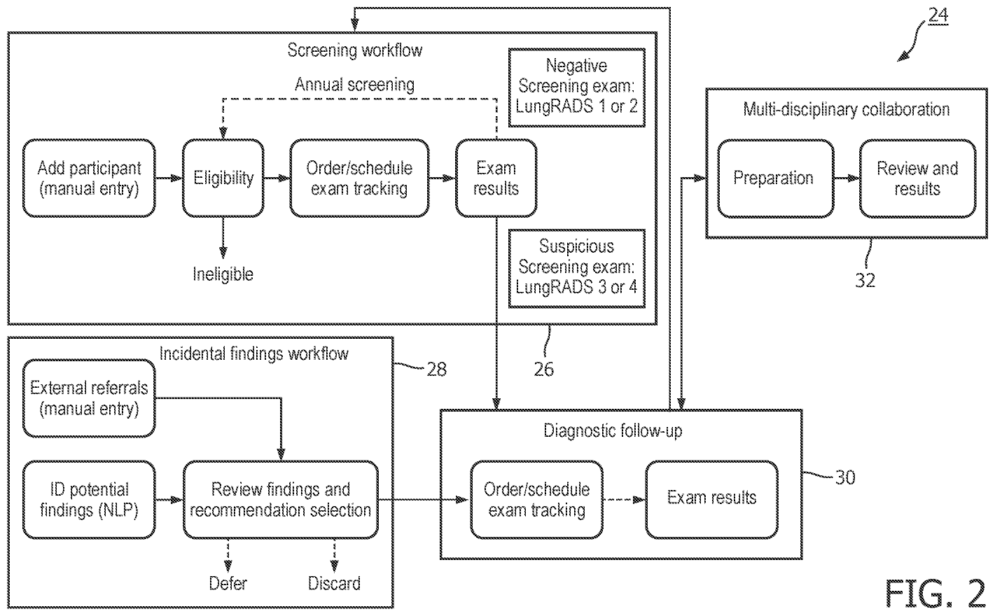

Referring now to FIG. 2, shown is a schematic diagram that illustrates example workflows in a lung nodule management application 24, in accordance with an embodiment of the invention. That is, FIG. 2 is illustrative of an example lung nodule management application 24, from which the analytics application 10 extracts data captured in the workflows of the application. In this example, the lung nodule management application 24 comprises a screening workflow 26, an incidental findings workflow 28, a diagnostic follow-up workflow 30, and a multidisciplinary collaboration workflow 32.

In the screening workflow 26, the patient management application 24 enables: adding patients to the worklist (manually or automatically), assessing their eligibility for lung cancer screening, ordering/scheduling exams and tracking their results, and making follow-up decisions. Depending on the outcome of the screening exam, patients may go through multiple rounds of annual screening.

In the incidental findings workflow 28, the patient management application 24 enables: adding patients with a possible incidental finding through the worklist (manually or automatically) and a review of their findings, making follow-up decisions and tracking exam results.

In the diagnostic follow-up workflow 30, the patient management application 24 enables, from patients that are either from the screening or incidental program, ordering/scheduling one or more diagnostic follow-up exams and tracking their results.

In the multidisciplinary collaboration workflow 32, the patient management application 24 enables: preparing for a multidisciplinary review and decision making through aggregation and entry of all exam results and patient information, review results and making decisions on diagnosis and treatment.

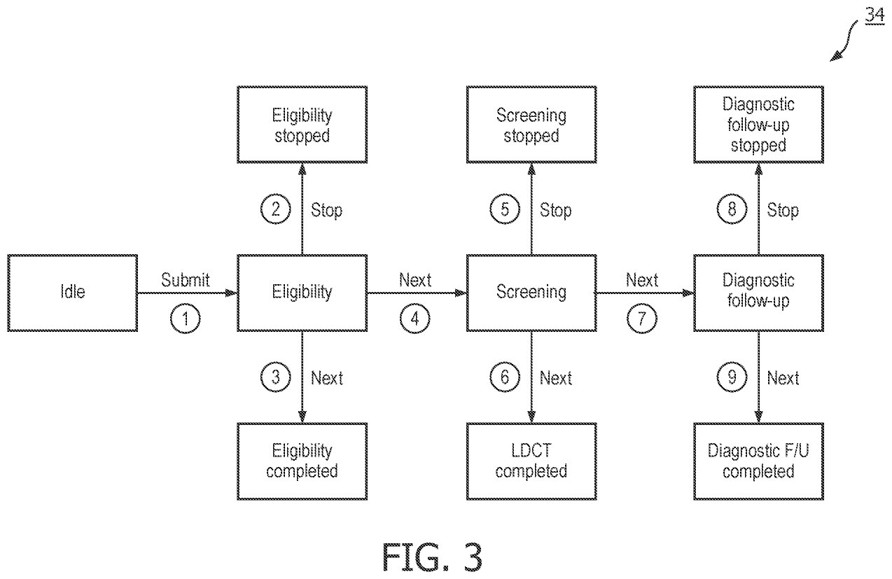

FIG. 3 is a schematic diagram that illustrates example main states (also, steps) of a lung (cancer) screening workflow 34, in accordance with an embodiment of the invention. Notably, the lung analytics data model describes the data captured in the workflows. In general, FIG. 3 reflects operations of the LCO application, which includes a lung cancer screening manager and an incidental nodule manager. The following describes the main states of the screening and incidental findings workflows (e.g., 26 and 28 from FIG. 2), or more generally, the steps in the lung cancer screening workflow. The main states of the lung cancer screening workflow 34 are depicted in FIG. 3, where the following user actions are defined: (1) enter a patient into a screening workflow and click submit; (2) stop the workflow in eligibility state; (3) proceed to the next screening cycle from the eligibility state (i.e., skip the current cycle); (4) click Next to go to the screening state; (5) stop the workflow in the screening state; (6) proceed to the next screening cycle from the screening state (i.e., skip the diagnostic follow-up); (7) click Next to go to the diagnostic follow-up; (8) stop the workflow in diagnostic follow-up; and (9) proceed to the next screening cycle from the diagnostic follow-up state. Explaining further, in step 1, potential participants in the lung cancer screening program are entered in a worklist. In the next step, the eligibility step, it is decided if the patient fulfils the criteria for inclusion in the screening program (step 3). If eligible, the baseline screening exam is ordered, scheduled and reviewed (steps 4 and 6). Depending on the result of the exam, the patient may either be selected for a next annual screening cycle (i.e. another exam, in case of a negative exam) or diagnostic follow-up (i.e. further investigation, in case of a positive exam) (next screening cycle: 1, 3, 4, 6 are repeated, diagnostic follow up: 7&9). In effect, FIG. 3 shows the main states of the screening workflow and all possible transitions between the states (i.e. proceeding to the next step). The user may stop the workflow in the various states (2, 5 & 8).

An ETL pipeline (e.g., ETL pipeline 14, FIG. 1) extracts information in any state of the workflow. For instance, the ETL pipeline may be required to show which patients are in state eligibility but have not been enlisted in screening yet. Or, the ETL pipeline may extract which patients were in state eligibility, but whose workflow has been stopped (e.g., meaning, the ETL pipeline should be able to extract the correct information in any of the states mentioned above).

In the lung screening workflow 34 depicted in FIG. 3, there are nine different states, but only six different paths. To test the workflow in all nine states, the following six scenarios may be exercised: (1) 1-test-2-test; (2) 1-3-test; (3) 1-4-test-5-test; (4) 1-4-6-test; (5) 1-4-7 test-8-test; (6) 1-4-7-9-test. The test scenarios test the robustness of the pipelines in extracting data from the lung cancer orchestrator workflows, providing a verification feature. Explaining further, the pipelines are extracting data from the workflows in the lung cancer orchestrator. In the test scenarios, the workflows are left in all possible states. For example: The consequence of leaving the workflow in the eligibility step is that the patient will not have had any screening exam. For instance, if the pipelines extract the data from this patient they will give back: #of screening exams=0. #of diagnostic follow-up tests=0. However, for patients that had a screening exam and diagnostic follow-up for that exam, the pipelines will give back: #of screening exams=1, diagnostic follow-up=True.

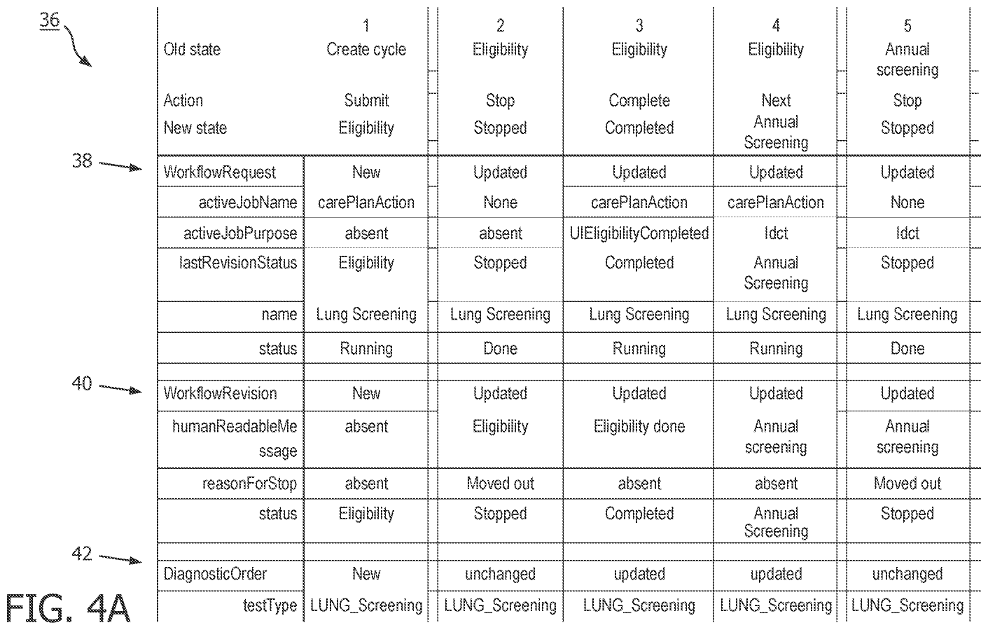

FIGS. 4A-4B are schematic diagrams that illustrates example entity tree objects and their creation/updates 36 in a lung screening process, in accordance with an embodiment of the invention. Note that the information depicted in FIG. 4B is an extension of the information depicted in FIG. 4A. FIGS. 4A-4B show the workflow request 38, workflow revision 40 and diagnostic order objects 42 in the entity tree and how they are created or updated during the nine steps mentioned above. In effect, FIGS. 4A-4B show what workflow objects get updated upon which actions in the application. Through this depiction, it is determined when and how the pipelines are triggered (e.g., trigger points) based on changes in objects in the entity tree in order to work in a robust way. From this table, it follows that the ETL process needs to monitor either the workflow request 38 or the workflow revision 40 for changes. The diagnostic order object 42 is not updated when the workflow is stopped. The workflow request 38 may be taken as a root object to identify the latest workflow revision.

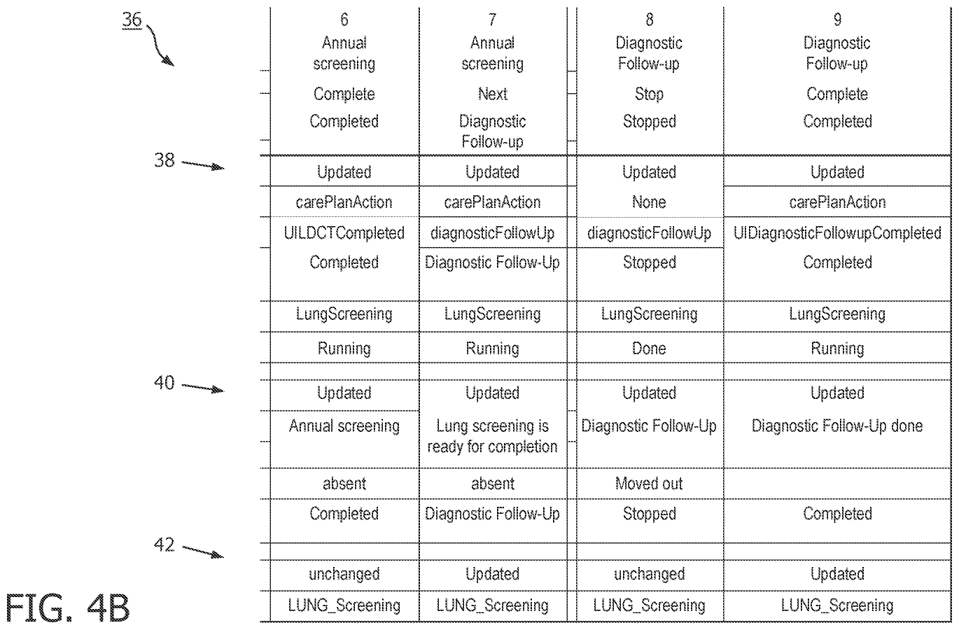

FIG. 5 is a schematic diagram that illustrates example main states (steps) of a lung incidental findings workflow 44, in accordance with an embodiment of the invention. Similar to FIG. 3, FIG. 5 describes operations of the LCO application, and in particular, the steps in the lung cancer incidental findings workflow. The following user actions are defined: (1) enter a patient into a lung incidental workflow and click submit; (2) stop the workflow in new findings state; (3) discard the finding and complete the workflow; (4) click Next to go to the diagnostic follow-up; (5) stop the workflow in diagnostic follow-up; (6) complete the workflow—no follow-up; and (7) proceed to screening from the diagnostic follow-up state. Explaining further, patients with an incidental finding in the lungs are entered into a worklist. This may be done in two ways: i) through a natural language processing algorithm searching through the radiology reports for a lung nodule finding, and/or ii) manually (step 1). In the new findings step, all new findings will be reviewed and a decision on the next step is taken (step 1). If the findings are regarded as not suspicious or a false positive, the findings may be discarded (step 3). If the findings are a true finding, diagnostic follow-up (additional investigation) may be ordered (steps 4, 6, 7). FIG. 5 shows the possible transitions between the different steps in the workflow. At the various steps in the workflow, the workflow may also be stopped (steps 2 and 5). For the lung incidental workflow 44, there is no single root object that is modified for every possible user action of interest. Therefore, a WorkflowRequest is a root object, as it is at least updated on the major state changes. Besides, the ETL pipeline may be run on a regular basis (e.g., weekly, monthly, etc.) to make sure that missing changes propagate into the analytics database 16 (FIG. 1).

Attention is now directed to database tables that are defined for certain embodiments of the analytics application 10. The database tables comprise the tables in the analytics database that are populated based on operations of the ETL pipelines. A base table is defined with common data elements, along with specific database tables for specific workflows. These database tables may be augmented, or new database tables may be created in the future to build analytics features across application boundaries. The following includes a list of table names and description of information contained therein corresponding to the specific workflows.

| Table name | Title |
|---|---|
| lung_screening_events | Contains information on patient data, workflow |
| | information and screening event data |
| lung_screening_diagnostic_followup_events | Contains information on diagnostic follow-up |
| | events for screening workflow |
| lung_incidental_events | Contains information on patients in the lung |
| | incidental workflow |
| lung_incidental_diagnostic_followup_events | Contains information on diagnostic follow-up |
| | events for incidental workflow |

One embodiment of an example base table is illustrated immediately below, where it is understood that all lung analytics database tables have the following columns in common:

| | Column Name | Type | Constraint | Description |
|---|---|---|---|---|
| 1 | logical_id | text | Primary Key | Unique ID for the event |
| 2 | last_updated | timestamptz | Not Null | The last updated time stamp of the event |
| | | | | information |
| 3 | organization_id | text | Not Null | Unique id for the organization |
| 4 | facility_id | text | | Unique id for the facility in the organization |
| 5 | etl_job_id | text | Not Null | Unique id for the specific ETL execution |
| 6 | etl_date | timestamptz | Not Null | Date and time when this record was |
| | | | | created/last updated |
| 7 | content | jsonb | | Reserved for future extensions |

The following example table defines the columns of the lung screening events table in the analytics database.

| | Column Name | Type | Constraint | Description |
|---|---|---|---|---|
| 1-7 | <standard> | <. . .> | <. . .> | See base table |
| 8 | patient_id | text | | Unique (ISPM) id for the patient |
| 9 | patient_mrn | text | | Organization specific Medical |
| | | | | Record Number for the patient |
| 10 | workflow_step | text | | Date and time when the screening |
| | | | | (using LDCT) took place |
| 11 | workflow_stopped | boolean | | Workflow status if it's stopped or not |
| 12 | workflow_stopped_reason | text | | Reason for Stopped workflow |
| 13 | workflow_revision_id | text | Not | Latest workflow revision id of |
| | | | Null | workflow |
| 14 | observation_smoking_cessation | text | | Smoking cessation status for patient |
| 15 | screening_event_id | text | | Latest event id of Order Information |
| 16 | screening_order_category_code | text | | Category code of Order Information |
| 17 | screening_order_category_display | text | | Category display of Order |
| | | | | Information |
| 18 | screening_event_category_code | text | | Category code of Event |
| 19 | screening_event_category_display | text | | Category display of Event |
| 20 | screening_event_group_code | text | | Capture group code of Event |
| 21 | screening_event_group_display | text | | Capture display code of Event |
| 22 | screening_date | timestamp | | Date and time when screening |
| | | | | (using LDCT) took place |
| 23 | screening_lung_rads_score_code | text | | Lung Rads score captured for |
| | | | | screening |
| 24 | screening_lung_rads_score_display | text | | Lung Rads score captured for |
| | | | | screening |
| 25 | screening_ct_other_findings_code | text | | Other findings score captured for |
| | | | | screening |
| 26 | screening_ct_other_findings_display | text | | Other findings score captured for |
| | | | | screening |
| 27 | screening_ct_examresult_modifier_S_code | text | | Lung RADS modifier S value |
| | | | | captured for screening |
| 28 | screening_ct_examresult_modifier_S_display | | | Lung RADS modifier S value |
| | | | | captured for screening |
| 29 | organization_name | text | | Patient belong to organization |
| 30 | facility_name | text | | Patient belong to facility |
| 31 | practitioner_name | text | | Patient record created/modified by |
| | | | | practitioner |
| 32 | practitioner_id | text | | Patient record created/modified by |
| | | | | practitioner |

The example table below defines the columns of the lung diagnostic follow-up events table for screening workflow

| | Column Name | Type | Constraint | Description |
|---|---|---|---|---|
| 1-7 | <standard> | <. . .> | <. . .> | See base table |
| 8 | workflow_revision_id | text | Not Null | Latest workflow |
| | | | | revision id of |
| | | | | workflow |
| 9 | workflow_request_id | text | Not Null | Latest workflow id of |
| | | | | workflow |
| 10 | order_category_code | text | | Category code of |
| | | | | Order Information |
| 11 | order_category_display | text | | Category display of |
| | | | | Order Information |
| 12 | event_category_code | text | | Category code of |
| | | | | Event |
| 13 | event_category_display | text | | Category display of |
| | | | | Event |
| 14 | event_group_code | text | | Capture group code |
| | | | | of Event |
| 15 | event_group_display | text | | Capture display code |
| | | | | of Event |
| 16 | pathology_event_technique_code | text | | Capture pathology |
| | | | | event |
| | | | | sub-categorization |
| 17 | pathology_event_technique_display | text | | Capture pathology |
| | | | | event |
| | | | | sub-categorization |
| 18 | pathology_event_tissuediagnosis_code | text | | Capture pathology |
| | | | | event technique |
| 19 | pathology_event_tissuediagnosis_code | text | | Capture pathology |
| | | | | event technique |

The following example table defines the columns of the lung incidental event table.

| | Column Name | Type | Constraint | Description |
|---|---|---|---|---|
| 1-7 | <standard> | <. . .> | <. . .> | See base table |
| 8 | incidental_event_date | timestamptz | | Date of event which |
| | | | | triggered the incidental |
| | | | | finding workflow |
| 9 | incidental_nlp_type | text | | Indicates whether found by |
| | | | | NLP |
| 10 | workflow_revision_id | text | Not Null | Latest workflow id of |
| | | | | workflow revision |
| 11 | incidental_category_name | text | | Category name of triggering |
| | | | | event |
| 12 | incidental_category_code | text | | Category code of triggering |
| | | | | event |
| 13 | decision_date | timestamptz | | Date of decision |
| 14 | decision_reference | text | | Normalized decision |
| 15 | decision_display | text | | User-facing text of decision |
| 16 | patient_id | text | | Unique (ISPM) id for the |
| | | | | patient |
| 17 | patient_mrn | text | | Organization specific |
| | | | | Medical Record Number for |
| | | | | the patient |
| 18 | workflow_step | text | | Date and time when the |
| | | | | screening (using LDCT) took |
| | | | | place |
| 19 | workflow_stopped | boolean | | Workflow status if it's |
| | | | | stopped or not |
| 20 | workflow_stopped_reason | text | | Reason for Stopped |
| | | | | workflow |
| 21 | organization_name | text | | Patient belong to |
| | | | | organization |
| 22 | facility_name | text | | Patient belong to facility |
| 23 | practitioner_name | text | | Patient record |
| | | | | created/modified by |
| | | | | practitioner |
| 24 | practitioner_id | text | | Patient record |
| | | | | created/modified by |
| | | | | practitioner |

The following table defines the columns of the lung diagnostic follow-up events for Incidental workflow table in the analytics database.

| | Column Name | Type | Constraint | Description |
|---|---|---|---|---|
| 1-7 | <standard> | <. . .> | <. . .> | See base table |
| 8 | workflow_revision_id | text | Not Null | Latest workflow |
| | | | | revision id of workflow |
| 9 | workflow_request_id | text | Not Null | Latest workflow id of |
| | | | | workflow |
| 10 | order_category_code | text | | Category code of |
| | | | | Order |
| | | | | Information |
| 11 | order_category_display | text | | Category display of |
| | | | | Order Information |
| 12 | event_category_code | text | | Category code of |
| | | | | Event |
| 13 | event_category_display | text | | Category display of |
| | | | | Event |
| 14 | event_group_code | text | | Capture group code of |
| | | | | Event |
| 15 | event_group_display | text | | Capture display code |
| | | | | of Event |
| 16 | pathology_event_technique_code | text | | Capture pathology |
| | | | | event |
| | | | | sub-categorization |
| 17 | pathology_event_technique_display | text | | Capture pathology |
| | | | | event |
| | | | | sub-categorization |
| 18 | pathology_event_tissuediagnosis_code | text | | Capture pathology |
| | | | | event technique |
| 19 | pathology_event_tissuediagnosis_code | text | | Capture pathology |
| | | | | event technique |

Data base creation scripts are used to create the database tables, and may have the following form:

```
    CREATE TABLE public.lung_screening_events (
  logical_id text NOT null primary key,
  last_updated timestamptz NOT null,
  organization_id text NOT null,
  facility_id text,
  etl_job_id text NOT null,
  etl_date timestamptz NOT null,
  “content” jsonb,
  workflow_revision_id text NOT null,
  workflow_step text,
  workflow_stopped bool,
  workflow_stopped_reason text,
  observation_smoking_cessation text,
  organization_name text,
  facility_name text,
  practitioner_id text,
  practitioner_name text,
  patient_mrn text,
  patient_id text,
  screening_event_id text,
  screening_order_category_code text,
  screening_order_category_display text,
  screening_event_category_code text,
  screening_event_category_display text,
  screening_event_group_code text,
  screening_event_group_display text,
  screening_date timestamptz,
  screening_lung_rads_score_code text,
  screening_lung_rads_score_display text,
  screening_ct_other_findings_code text,
  screening_ct_other_findings_display text,
  screening_ct_examresult_modifier_s_code text,
  screening_ct_examresult_modifier_s_display text
);
```

```
    CREATE TABLE
    public.lung_screening_diagnostic_followup_events (
  logical_id text NOT null primary key,
  last_updated timestamptz NOT null,
  etl_job_id text NOT null,
  etl_date timestamptz NOT null,
  “content” jsonb,
  workflow_request_id text NOT null,
  workflow_revision_id text NOT null,
  order_category_code text,
  order_category_display text,
  event_category_code text,
  event_category_display text,
  event_group_code text,
  event_group_display text,
  pathology_event_technique_code text,
  pathology_event_technique_display text,
  pathology_event_tissuediagnosis_code text,
  pathology_event_tissuediagnosis_display text
);
```

```
  CREATE TABLE public.lung_incidental_events (
  logical_id text NOT NULL PRIMARY KEY,
  last_updated timestamptz NOT NULL,
  organization_id text NOT NULL,
  facility_id text,
  etl_job_id text NOT NULL,
  etl_date timestamptz NOT NULL,
  “content” jsonb,
  workflow_revision_id text NOT NULL,
  workflow_step text,
  workflow_stopped bool,
  workflow_stopped_reason text,
  organization_name text,
  facility_name text,
  practitioner_id text,
  practitioner_name text,
  patient_mrn text,
  patient_id text,
  decision_date timestamptz,
  incidental_event_date timestamptz,
  incidental_event_category_code text,
  incidental_event_category_name text,
  incidental_event_nlp_type text,
  decision_reference text,
  decision_display text,
  decision_recommendation text
);
```

```
    CREATE TABLE
    public.lung_incidental_diagnostic_followup_events (
  logical_id text NOT null primary key,
  last_updated timestamptz NOT null,
  etl_job_id text NOT null,
  etl_date timestamptz NOT null,
  “content” jsonb,
  workflow_request_id text NOT null,
  workflow_revision_id text NOT null,
  order_category_code text,
  order_category_display text,
  event_category_code text,
  event_category_display text,
  event_group_code text,
  event_group_display text,
  pathology_event_technique_code text,
  pathology_event_technique_display text,
  pathology_event_tissuediagnosis_code text,
  pathology_event_tissuediagnosis_display text
);
```

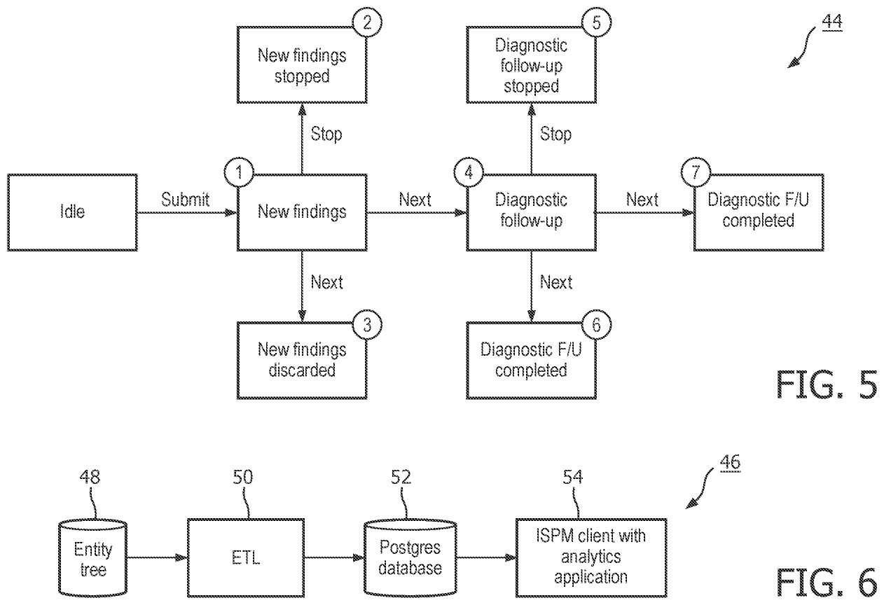

FIG. 6 is a schematic diagram that illustrates an example overall, high level design of an analytics application 46 with ETL pipeline in a cloud based software as a service system, in accordance with an embodiment of the invention, and includes (as similarly described above) an entity tree 48, ETL pipeline 50, Postgres (e.g., relational, though not limited to Postgres databases) database 52, and ISPM client with analytics application 54. Focusing on the ETL pipeline 50, the ETL pipeline 50 is configured to extract, transform, and load data into the analytics database (e.g., the data structures described above for the analytics database). The high-level design of the analytics application 46 with ETL pipeline 50 is as follows. The ISPM entity tree 48 contains data relevant to lung analytics. A periodic ETL process 50 extracts data from the entity tree 48. This extracted data is stored in the Postgres database 52 (called the analytics database). The analytics application runs in the ISPM client 54 and displays statistics.

The ETL pipeline 50 comprises three steps: (1) Extract: fetch objects from the entity tree 48; (2) Transform: create NiFi FlowFile attributes from these objects; and (3) Load: insert records filled with these attributes into the analytics database 52. Note that the objects themselves are defined in the entity tree 48. The objects that are fetched are described in the NiFi pipeline. In other words, the objects are not defined in the NiFi pipeline, but are used in the pipeline to describe analytical behaviors associated to it and therein named as attributes. Additionally, the transformation is from the data in the lung nodule management program to a format suitable for populating the database structures of the analytics database. It should be appreciated by one having ordinary skill in the art that there may be some additional cleaning and normalization performed. Expanding upon these steps, the extraction description below explains the structure of the relevant entity tree objects and how to retrieve them (e.g., via REST calls).

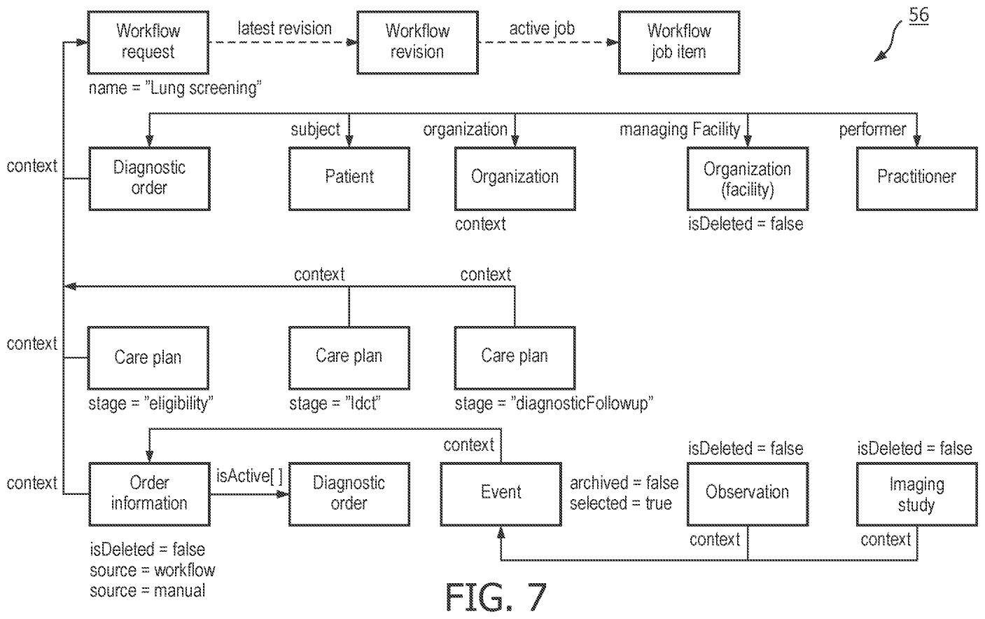

FIG. 7 is a schematic diagram that illustrates example relevant entity tree objects 56 for a lung analytics ETL pipeline, in accordance with an embodiment of the invention. Notably, the entity tree objects 56 are largely available as part of the LCO application and IntelliSpace Precision Medicine Platform. FIG. 7 shows objects in the entity tree that are relevant to the lung analytics ETL pipeline. Generally, to extract analytical insights that are specific for the lung cancer screening application, the pipelines need to specifically monitor if there is change in that application (e.g., a trigger point). Therefore, it is specified when the pipelines need to be triggered and fetch the updated workflow statuses and new data entered in the application. This is done through monitoring a specific object in the entity tree called the workflow request object with the name ‘Lung Screening’. A further contextual specification of this object is called a diagnostic order object, which provides information on the patient, organization, facility, and practitioner. From this, it can be derived in which hospital and hospital facility and for which particular patient the workflow status changed and thus from where the extracted data originate.

As depicted in FIG. 7, the root object is a workflow request object with name=“Lung Screening”, and associated with this is a latest workflow revision object and a set of workflow job items. Referring to the workflow request object in its context is a diagnostic order object, from which a patient, organization, facility and a practitioner object can be derived. Each step in the lung screening workflow ends with a care plan object. The initial screening event is modelled as an order information object, a diagnostic order object and an event, and so is each diagnostic follow-up study. With regard to fetching entity tree objects, the table below further specifies the entity tree objects mentioned above, where the table defines how to navigate from one entity tree object to another.

| Object name | Object type | Retrieval |
|---|---|---|
| workflowRequestObj | WorkflowRequest | ${ET}/WorkflowRequest?name=Lung Screening |
| incidentalWorkflowRequestObj | WorkflowRequest | ${ET}/WorkflowRequest?name=Lung Incidental |
| workflowJobItemObj | WorkflowJobItem | ${ET}/ WorkflowJobItem?id=${ workflowRequestObj.revisions[-1].activeJobId } |
| diagnosticOrderObj | DiagnosticOrder | ${ET}/DiagnosticOrder?workflowRequest=${ |
| | | workflowRequestObj.id } |
| organizationObj | Organization | id = diagnosticOrderObj.resource.organization |
| | | ${ET}/Organization/${id} |
| facilityObj | Facility | id = diagnosticOrderObj.resource.managingFacility |
| | | ${ET}/Organization/${id} |
| patientObj | Patient | id = diagnosticOrderObj.resource.subject |
| | | ${ET}/Patient/${id} |
| practitionerObj | Practitioner | id = diagnosticOrderObj.resource.performer |
| | | ${ET}/Practitioner/${id} |
| smokingObj | Observation | patientID = diagnosticOrderObj.resource.subject |
| | | workflowRequestID = |
| | | diagnosticOrderObj.resource.workflowRequest |
| | | ${ET}/Observation?subResourceType= |
| | | RISK_FACTORS_SOCIAL_HISTORY&context=${patientID}&context= |
| | | ${workflowRequestID} |
| screeningOrderInformationObj | OrderInformation | workflowRevisionId = workflowRequestObj.revisions[-1].id |
| | | ${ET}/OrderInformation?context=${workflowRevisionId]&source= |
| screeningEventObj | Event | workflowid=screeningOrderInformationObj.resource.context.reference |
| | | where context.resourceType==“validatedEvent” |
| | | ${ET}/Event/${id} |
| incidentalEventObj | Event | Dynamic search for events related to an |
| | | incidentalWorkflowRequestObj |
| | | The incidentalEventObj is the object with the oldest creation |
| | | date |
| carePlanObj | CarePlan | Find the CarePlan CP object with type = |
| | | “LungIncidentalDecisionCapture” and stage.code = |
| | | “newFindings” that refers to the latest revision of WR in its |
| | | context. Note that if a decision was saved and then |
| | | subsequently deleted, an empty care plan object remains that |
| | | will be used to record the new decision once provided. Such an |
| | | empty care plan object will not have the priority information |
| | | shown below, and should be ignored. |
| diagnosticFollowUpOrderInformationObj | OrderInformation | workflowRevisionId = workflowRequestObj.revisions[-1].id |
| | | ${ET}/OrderInformation?source=manual&statusCode= |
| | | completed&context=${workflowRevisionId} |
| diagnosticFollowUpEventObj | Event | id= |
| | | diagnosticFollowUpOrderInformationObj.resource.context. |
| | | reference where context.resourceType==“validatedEvent” |
| | | ${ET}/Event/${id} |

The following section describes the transformation from fields in the entity tree objects to columns in the analytics database tables. The table below describes the location in the ISPM's entity tree database from where each of the data elements in the pathways analytics database is extracted. The “Retrieval” column describes the resources in the Entity Tree where these data objects may be found. In other words, the “Retrieval” column in this table specifies the specific object from the entity tree that is fetched to populate the lung analytics database table. The ETL pipeline, built in one embodiment using Apache NiFi, connects to the entity tree and retrieves these data elements.

All lung analytics database tables have the following columns of a base table in common:

| | Column Name | Retrieval |
|---|---|---|
| 1 | logical_id | workflowRequestObj.id |
| 2 | last_updated | The last updated time stamp of the event |
| | | information |
| 3 | organization_id | organizationObj.id |
| 4 | facility_id | facilityObj.id |
| 5 | etl_job_id | Unique id for the specific ETL execution |
| 6 | etl_date | Date and time when this record was created/last |
| | | updated |
| 7 | content | Reserved for future extensions |

The following (lung screening workflow) table defines the columns of the lung screening events table in the analytics database.

| | Column Name | Retrieval |
|---|---|---|
| 1-7 | <standard> | |
| 8 | patient_id | patientObj.id |
| 9 | patient_mrn | patientObj.identifier[0].MRN |
| 10 | workflow_step | workflowJobItemObj. purpose |
| 11 | workflow_stopped | workflowRequestObj.latestRevisionStatus |
| 12 | workflow_stopped_reason | workflowRequestObj.revisions[-1].reasonForStop |
| 13 | workflow revision id | workflowRequestObj.revisions[-1].id |
| 14 | observation_smoking_cessation | smokingObj.resource.smokingCessationCounselling.display |
| 15 | screening_event_id | screeningOrderInformationObj.resource.context.reference |
| | | where |
| | | context.resourceType==“validatedEvent” |
| 16 | screening_order_category_code | screeningOrderInformationObj.resource.category.code |
| 17 | screening_order_category_display | screeningOrderInformationObj.resource.category.display |
| 18 | screening_event_category_code | screeningEventObj.category.code |
| 19 | screening_event_category_display | screeningEventObj.category.display |
| 20 | screening_event_group_code | screeningEventObj.group.code |
| 21 | screening_event_group_display | screeningEventObj.group.display |
| 22 | screening_date | screeningEventObj.content[0].data.dateOfProcedure |
| | | \|\| screeningEventObj.date |
| 23 | screening_lung_rads_score_code | screeningEventObj.content[0].data.cTExamResultByLungRADSCategory.display |
| 24 | screening_ct_other_findings_code | screeningEventObj.content[0].data.otherFindings.display |
| 25 | screening_ct_examresult_modifier_S_code | screeningEventObj.content[0].data.ctExamResultWith ModifierS.display |
| 26 | organization_name | organizationObj.name |
| 27 | facility_name | facilityObj.name |
| 28 | practitioner_name | practitionerObj.name |
| 29 | practitioner_id | practitionerObj.id |
| 30 | screening_lung_rads_score_display | screeningEventObj.content[0].data.cTExamResultByLungRADSCategory.display |
| 31 | screening_ct_other_findings_display | screeningEventObj.content[0].data.otherFindings.display |
| 32 | screening_ct_examresult_modifier_S_display | screeningEventObj.content[0].data.ctExamResultWithModifierS.display |

The following (lung diagnostic follow-up events table for screening workflow) table defines the columns of the lung diagnostic follow-up events table in the analytics database.

| | Column Name | Retrieval |
|---|---|---|
| 1 | event_id | diagnosticFollowUpOrderInformationObj.resource.context.reference |
| | | where |
| | | context.resourceType==“validatedEvent” |
| 2-7 | <standard> | See section 1.2.1. |
| 8 | workflow_revision_id | workflowRequestObj.revisions[-1].id |
| 9 | event_id | diagnosticFollowUpOrderInformationObj.resource.context.reference |
| | | where |
| | | context.resourceType==“validatedEvent” |
| 10 | order_category_code | diagnosticFollowUpOrderInformationObj.resource.category.code |
| 11 | order_category_display | diagnosticFollowUpOrderInformationObj.resource.category.display |
| 12 | event_category_code | diagnosticFollowUpEventObj.category.code |
| 13 | event_category_display | diagnosticFollowUpEventObj.category.display |
| 14 | event_group_code | diagnosticFollowUpEventObj.group.code |
| 15 | event_group_display | diagnosticFollowUpEventObj.group.display |
| 16 | pathology_event_technique_code | diagnosticFollowUpEventObj.data.technique.code |
| 17 | pathology_event_technique_display | diagnosticFollowUpEventObj.data.technique.display |
| 18 | pathology_event_tissuediagnosis_display | diagnosticFollowUpEventObj.data.tissuediagnosis.code |
| 19 | pathology_event_ tissuediagnosis_display | diagnosticFollowUpEventObj.data.tissuediagnosis.display |

The table below is the lung incidental events table.

| | Column Name | Retrieval |
|---|---|---|
| 1-7 | <standard> | See base table |
| 8 | incidental_event_ date | incidentalEventObj.date |
| 9 | incidental_nlp_type | incidentalEventObj.nlpFindings.nlpPositiveFindings and |
| | | incidentalEventObj.nlpFindings.nlpType.code==‘lung’ |
| 10 | workflow_revision_id | Latest workflow revision id of workflow |
| 11 | incidental_category_name | incidentalEventObj.category.display |
| 12 | incidental_category_code | incidentalEventObj.category.code |
| 13 | decision_date | incidentalWorkflowRequestObj.revisions[-1].items[0].meta.lastUpdated |
| | | IF incidentalWorkFlow.RequestObj.revisions[-1].items[0].status != ‘Running’ |
| 14 | decision_reference | carePlanObj.priority[0].diagnosticPlanInfo.references[0].reference |
| 15 | decision_display | carePlanObj.priority[0].diagnosticPlanInfo.references[0].display |
| 16 | decision_recommendation | carePlanObj.priority[0].formData.recommendation |
| 16 | patient_id | patientObj.id |
| 17 | patient_mrn | patientObj.identifier[0].MRN |
| 18 | workflow_step | workflowJobItemObj. purpose |
| 19 | workflow_stopped | workflowRequestObj.latestRevisionStatus |
| 20 | workflow_stopped_reason | workflowRequestObj.revisions[-1].reasonForStop |
| 21 | organization_name | organizationObj.name |
| 22 | facility_name | facilityObj.name |
| 23 | practitioner_name | practitionerObj.name |
| 24 | practitioner_id | practitionerObj.id |

The following table (lung diagnostic follow-up events table for incidental workflow) defines the columns of the lung diagnostic follow-up events table in the analytics database.

| | Column Name | Retrieval |
|---|---|---|
| 1 | event_id | diagnosticFollowUpOrderInformationObj.resource.context.reference |
| | | where context.resourceType==“validatedEvent” |
| 2-7 | <standard> | See section 1.2.1. |
| 8 | workflow_revision_id | workflowRequestObj.revisions[-1].id |
| 9 | event_id | diagnosticFollowUpOrderInformationObj.resource.context.reference |
| | | where context.resourceType==“validatedEvent” |
| 10 | order_category_code | diagnosticFollowUpOrderInformationObj.resource.category.code |
| 11 | order_category_display | diagnosticFollowUpOrderInformationObj.resource.category.display |
| 12 | event_category_code | diagnosticFollowUpEventObj.category.code |
| 13 | event_category_display | diagnosticFollowUpEventObj.category.display |
| 14 | event_group_code | diagnosticFollowUpEventObj.group.code |
| 15 | event_group_display | diagnosticFollowUpEventObj.group.display |
| 16 | pathology_event_technique_code | diagnosticFollowUpEventObj.data.technique.code |
| 17 | pathology_event_technique_display | diagnosticFollowUpEventObj.data.technique.display |
| 18 | pathology_event_tissuediagnosis_display | diagnosticFollowUpEventObj.data.tissuediagnosis.code |
| 19 | pathology_event_ tissuediagnosis _display | diagnosticFollowUpEventObj.data.tissuediagnosis.display |

With regard to the configuration of the ETL pipeline, the following variables, which are specific to the analytics application embodiments, control the execution of the NiFi pipeline for pipeline analytics.

| Variable Name | Description |
|---|---|
| Intellispace-Authorization | A value that allows access to navigations for all organizations |
| database_connection_url | The analytics database connection string, containing the IP, port, |
| | username and password. |
| database_driver_path | The location of the database driver inside the docker |
| database_schema | The name of the schema that contains the pathway analytics |
| | tables |
| database_table | The name of the analytics database table i.e. |
| | lung_screening_events, lung_diagnostic_followup_events |
| entity_tree_url (ET) | The URL (including port) that provides access to the entity tree |
| | service |
| max_record_count | The maximum number of records to fetch from the entity tree in |
| | one query |
| start_date | The date where the ETL should start when the database is empty |
| | (e.g. ‘2018-01-01T00:00:00.000+00:00’) |
| window_size_in_msecs | The time window size for a single query to the entity tree (one day = |
| | 86400000 msecs) |
| test_name | The constant value “Lung Screening”/”Lung Incidental” |

Although one embodiment uses variables to control the NiFi pipelines, in some embodiments, the pipeline variables may be replaced by parameters. Notably, variable and parameter behavior changes depending on the context of NiFi in different scenarios. One difference between variables and parameters is that using parameters allows saving sensitive information like password, organization id, etc. (which is not possible using variables). Hence, in some embodiments, parameters may be used.

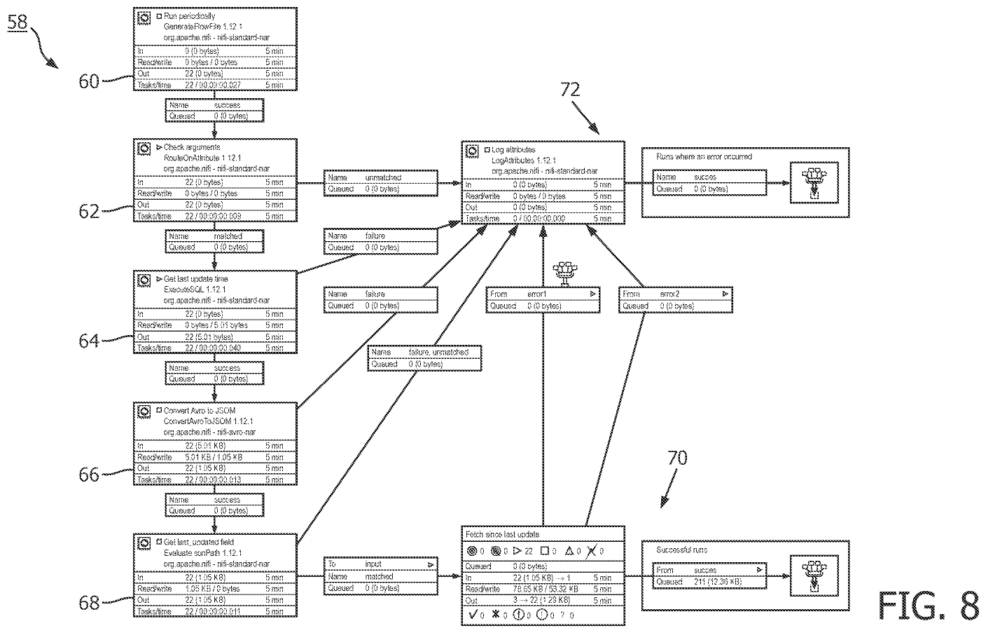

FIG. 8 is a schematic diagram that illustrates an example top-level ETL pipeline 58, in accordance with an embodiment of the invention. Note that the NiFi user interface provides mechanisms for creating dataflows, as well as visualizing, editing, monitoring, and administering those dataflows. FIG. 8 shows the use of different processors, connectors between processors, input/output port connectors, and sub-processor-groups (and also, the root processor group or NiFi template is called (not shown in FIG. 8)). Note that much of the individual data (e.g., bytes, times) depicted in each processor block is merely used for illustration, with emphasis placed primarily on identification and functionality of the main components of the ETL pipeline. Execution of the pipeline starts from the first processor, named Run periodically. Inside the Fetch since last Update sub-processor group is the logic related to ETL. Once started, the ETL pipeline 58 runs periodically. On each run, if an error occurs, then the error is logged and that run stops (but this does not disable the periodic repetition). In the next period, the ETL pipeline 58 runs again and starts from the last successful insertion into the analytics database. If the cause of the problem is not solved, then the pipeline fails again. Note that the ETL pipeline 58 may be used to retrieve historic data and/or to do an incremental update since the last run.

In the description that follows, each of the processors depicted in FIG. 8 are described in more detail. NiFi provides a processor configuration window, which has multiple sub-menus. It is noted that, where possible, time stamp strings are standardized to the ISO-8601 format (′yyyy-MM-ddTHH:mm:ss.SSSXX where XX represents the time zone relative to UTC as either ‘+hh:mm’ or ‘−hh:mm’).

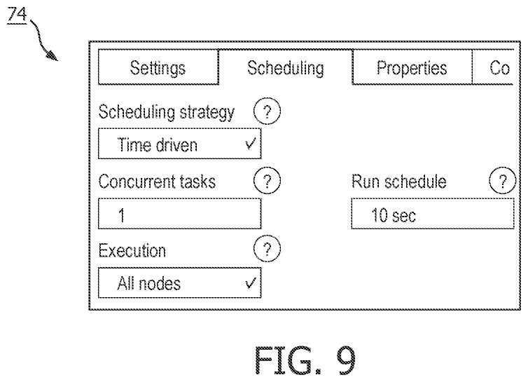

FIG. 9 is a schematic diagram that illustrates an example scheduling strategy 74 of a GenerateFlowFile processor 60 during development, in accordance with an embodiment of the invention. During testing, this processor 60 is programmed to run periodically (e.g., every ten seconds). In production, this processor 60 should be in CRON driven mode. In some embodiments, the processor 60 may be programmed to run every hour, or every night, etc., depending on the requirements. On each run, this processor 60 generates an empty FlowFile that triggers the rest of the pipeline.

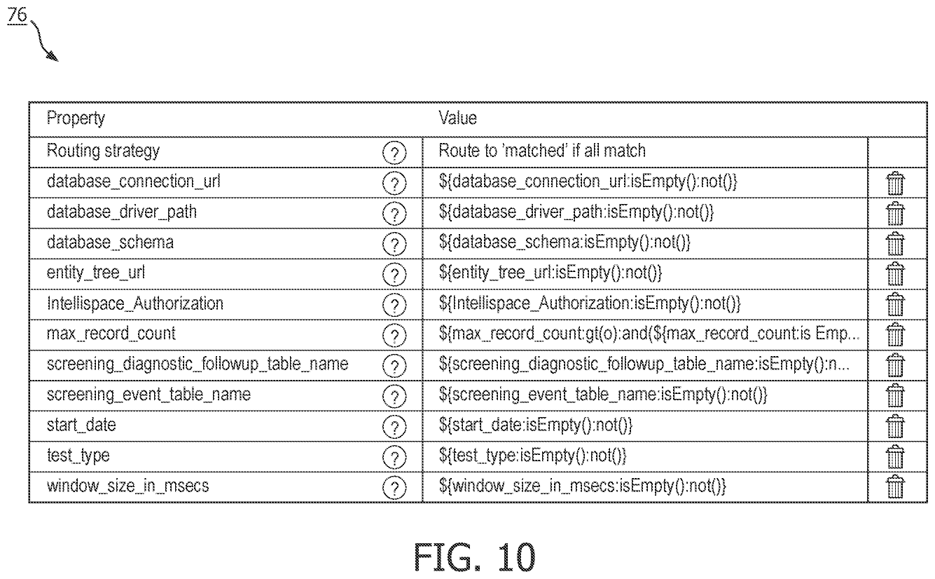

FIG. 10 is a schematic diagram that illustrates example checks 76 performed by a check arguments processor 62, in accordance with an embodiment of the invention. This processor 62 checks whether the configuration variables have appropriate values. As an example, the entity tree and database tables have a location where they are stored and maintained and a specific identifier number. If these are not found, the pipeline cannot fetch the data and is thus stopped (e.g., the pipeline is stopped if there is any deviation).

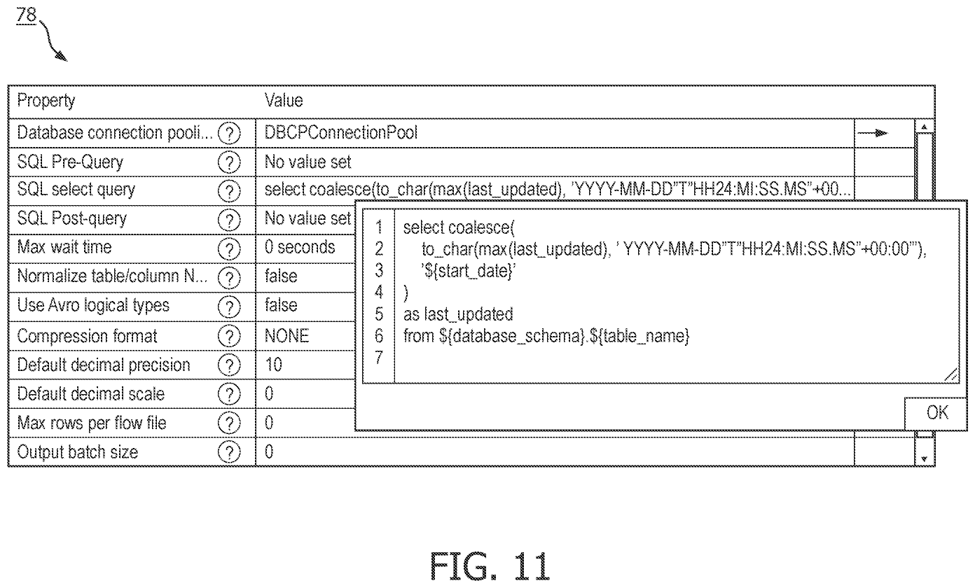

FIG. 11 is a schematic diagram that illustrates finding a last updated time stamp 78 for processor 64, in accordance with an embodiment of the invention. In FIG. 11, the sub-menu called properties of processor (Property) and its variables are displayed. Here their values can be defined. This processor 64 reads the last updated time stamp from the analytics database. If the database table is empty, then the configured start time is used. Note how “to_char” is used to force the time stamp into the standard ISO 8601 format. Note how “coalesce” is used to substitute the start date when the table is empty.

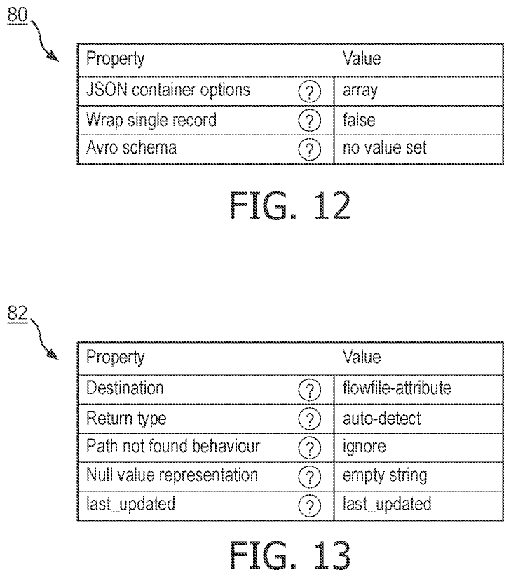

FIG. 12 is a schematic diagram that illustrates a processor 66 that comprises setting of an Avro to JSON converter 80, in accordance with an embodiment of the invention. This processor 66 converts the output of the previous processor from the Avro format into Json. No special settings are used.

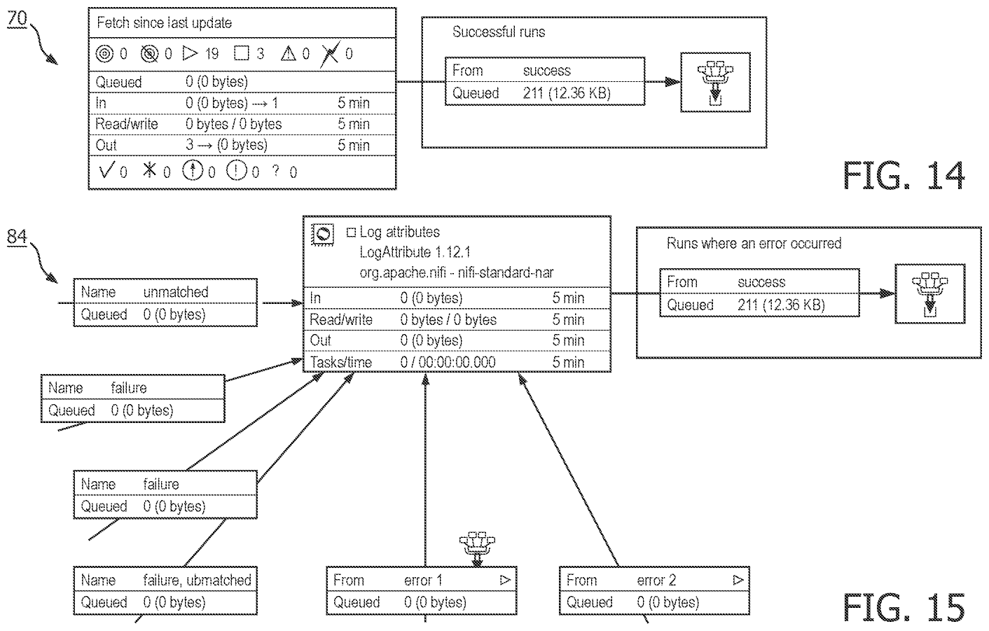

FIG. 13 is a schematic diagram that illustrates a processor 68 for storing last updated information from JSON content into a FlowFile attribute 82, in accordance with an embodiment of the invention. This processor 68 copies the last_updated field from the JSON content into an attribute of the same name.

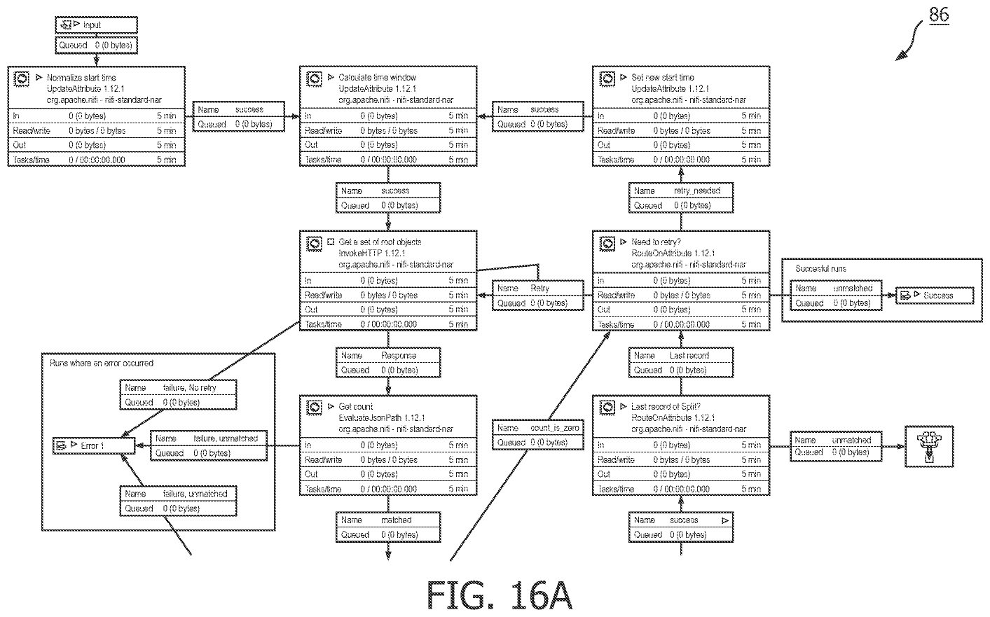

FIG. 14 is a schematic diagram that illustrates an example pipeline loop 70 with successful outputs, in accordance with an embodiment of the invention. This process group takes the last_updated FlowFile attribute, fetches all entity tree objects that have been created since that time stamp, and stores the relevant ones in the analytics database. In one embodiment, on successful completion, a FlowFile is output into the funnel. As is known, a funnel is a NiFi component that is used to combine the data from several Connections into a single Connection. In the content inside fetch since last update sub-processor group 70, there is logic related to ETL having several connectors, processor and sub-processor-group and the final result is aggregated into single connection as successful runs. From the output of the funnel, connections to different instance may be implemented depending on use cases. In some embodiments, the funnel as an ETL tool may be replaced with a counter to track the successful record count. On failure, attributes are logged, and an error is raised. This process group is discussed below.

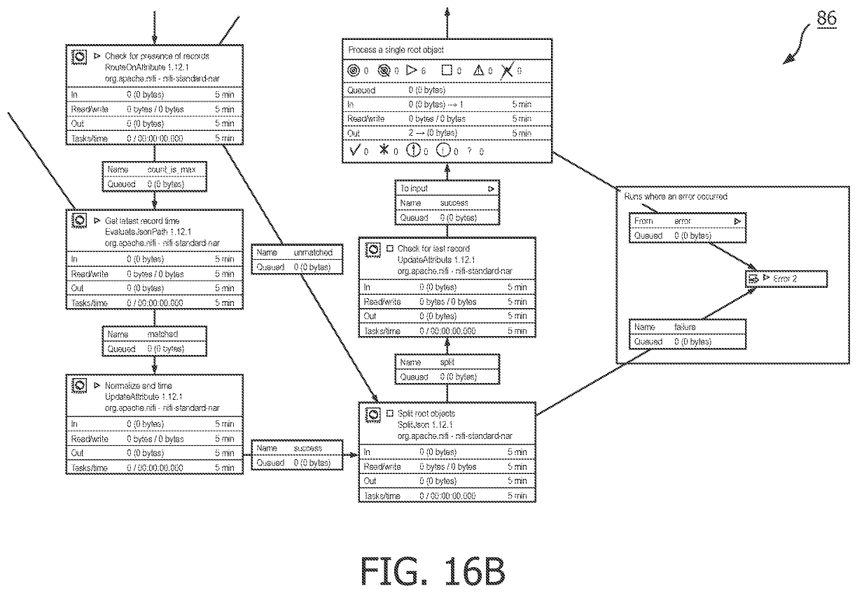

FIG. 15 is a schematic diagram that illustrates example error handling 84 in a main pipeline, in accordance with an embodiment of the invention. In case of an error in the main pipeline, this processor 72 logs all FlowFile attributes, routes to a funnel, and ends this run of the pipeline. Note that the periodic run is not disabled: the pipeline runs again at the time determined by the first processor (e.g., processor 60).

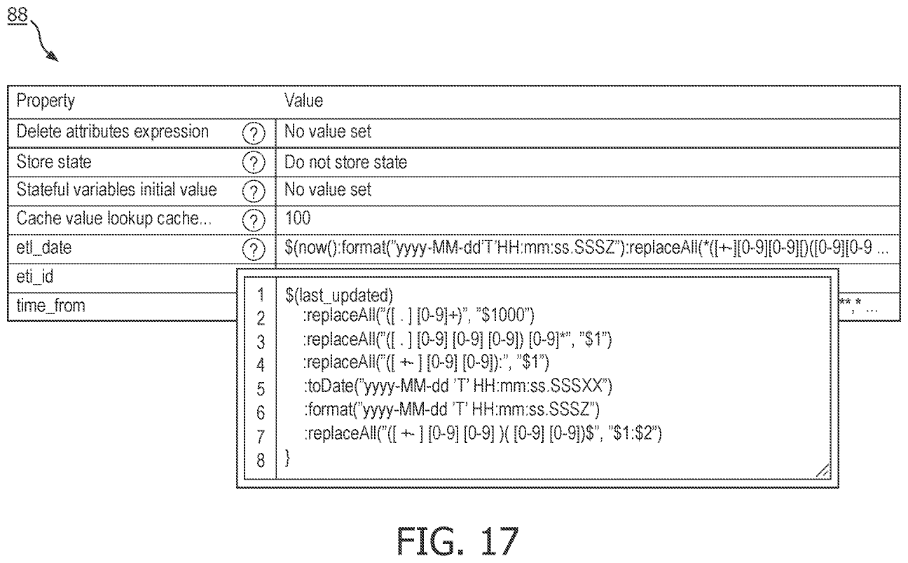

FIGS. 16A-16B are schematic diagrams that illustrate an example pipeline loop 86 that fetches root objects in chunks, in accordance with an embodiment of the invention. Note that the information in FIG. 16B is an extension of the information shown in FIG. 16A. This pipeline loop 86 is responsible for fetching all data since a specified last_updated time stamp. It is a loop because the number of records obtained in one query to the entity tree is limited by both a time window and a maximum record count. There is a maximum record count to prevent a network overload. There is a maximum time window to prevent the sort in the database (see below) from becoming very inefficient. The maximum record count and time window size may be set independently (e.g., dependent on the circumstances which of the two will limit the number of records returned). Explaining further, FIGS. 16A-16B depict the content of the sub-processor group called fetch since last update (FIG. 8), and performs some specific tasks as follows: normalize the start time, calculate time window, get the root object, get the count of entry, check the presence of records in the root object entry (when 0 records are in the entry, no processing of single root object; when record count equals max_count or in between 0<record count<max_count), normalize the end time, split and check for last record, process a single entry as FlowFile in NiFi ETL called as single root object, on last record Boolean value, move the processed record to success connector or unmatched connector, and evaluate a condition—i.e., check if no more entry left from entity tree until present date of execution (on false, execution is processed successfully on unmatched connector pointing to output port called success; on true, retry_needed connector and start normalizing the date again). This process continues until matching this latter condition and moving to an unmatched connector. Components depicted in FIGS. 16A-16B are described further below.

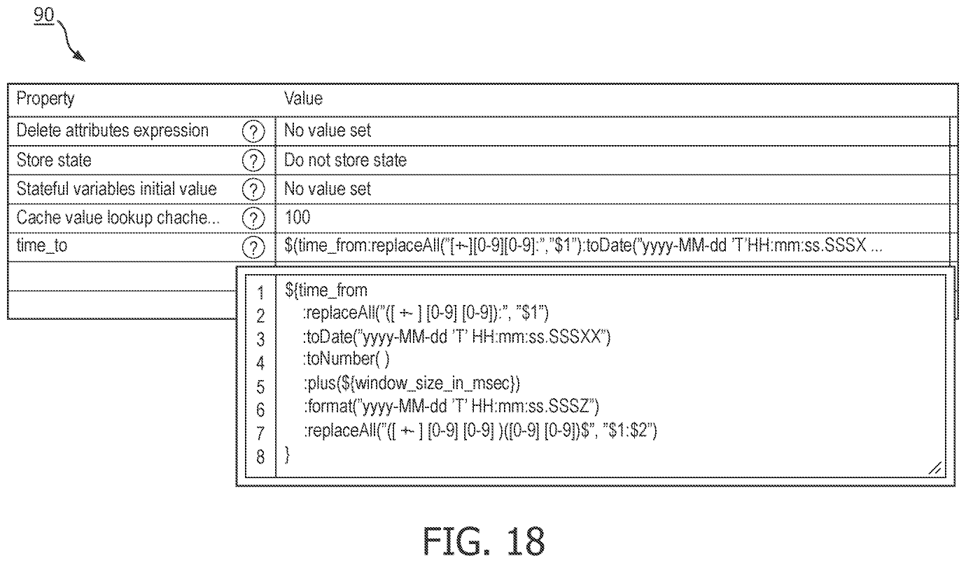

FIG. 17 is a schematic diagram that illustrates setting a start time to a normalized value of last_updated 88, in accordance with an embodiment of the invention. For instance, FIG. 17 shows how the start time of the window, time_from, is calculated from the last_updated attribute. This attribute contains either the time stamp of the most recent record in the analytics database table, or if the table is empty, the start time as configured. In one embodiment, the time stamp is normalized as follows: (1) First add three trailing zeros to the fractional part, and then keep the three leading digits. Trailing zeros are added since Java's SimpleDateTimeFormat interprets ‘12:1:1.1’ as ‘12:01:01:001’. This is because ‘SSS’ represents milliseconds, rather than fractions of a second. This is a known shortcoming of SimpleDateTimeFormat. In some implementations, there is a need to trim to three fractional digits (e.g., since the entity tree does not accept more); (2) Replace ‘+12:34’ by ‘+1234’, run it through toDate which then interprets the time zone correctly, and run it back through format, which returns the date/time string with a time zone 400:00′. From here on, all date/times objects are represented in UTC.

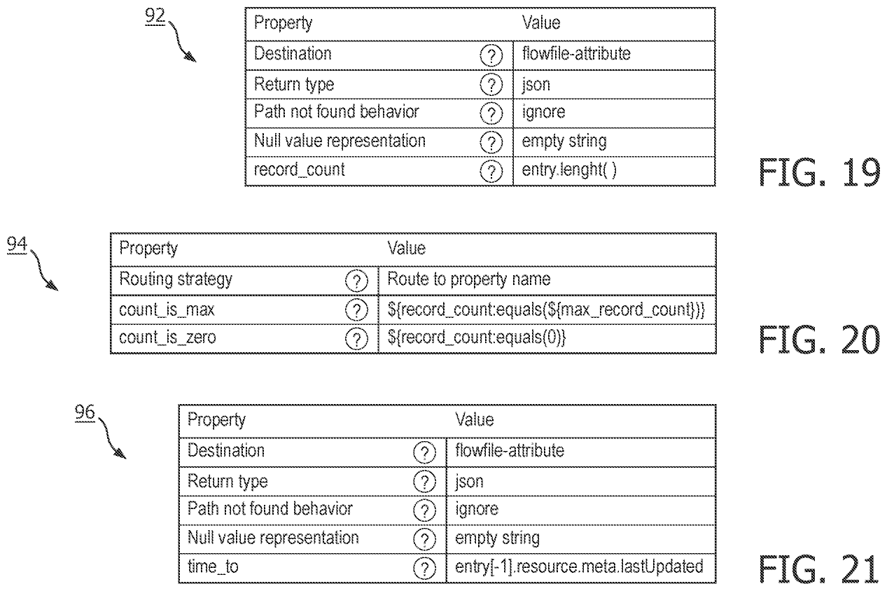

FIG. 18 is a schematic diagram that illustrates calculating an end time of a window by adding a window size to a start time 90, in accordance with an embodiment of the invention. That is, FIG. 18 shows how to calculate the end time of the time window, given the start time and the window size. In one embodiment, the calculation is as follows: (1) Convert the string representation of time_to to NiFi's internal date format; (2) Add the window size in milliseconds; and (3) Convert back to the standard string format.

With regard to the processor in FIG. 16A corresponding to getting a set of root objects, this processor retrieves a set of objects from the entity tree. The query is structured as follows:

${entity_tree_url}/WorkflowRequest?name=${test_type}&_sort:asc=timestamp&ti
mestamp=>${time_from:replace(‘+’,
‘%2b’)}@@timestamp=<${time_to:replace(‘+’,
‘%2b’)}&_count=${max_record_count}

The objects are sorted according to timestamp in ascending order, making sure the oldest max_record_count objects in the specified time window are retrieved first. If there are more objects in this time window, the time window is moved to start at the time stamp of the latest object thus retrieved. If all objects of this time window have been retrieved, then the time window is moved to start at the end of the previous window. Note that having a limited time window prevents the sort from being overloaded with, possibly, 100,000 objects when doing a historic fetch of all data. The time window should typically be set to one or a few days. It is further noted that the time_from is included in the search (using greater equal). For instance, if the search is started at 2018-01-01, an object that is dated ‘2018-01-01T00:00:00’ is included. Note also that time_end is also included in the search. If an object has the exact same time stamp as the end time of a window, it might be fetched twice (which is acceptable, as the database insert statement handles this). Additionally, it is noted that in some embodiments, ‘+’ signs are encoded as ‘%2b’ (otherwise they are replaced by spaces before they reach the entity tree server).

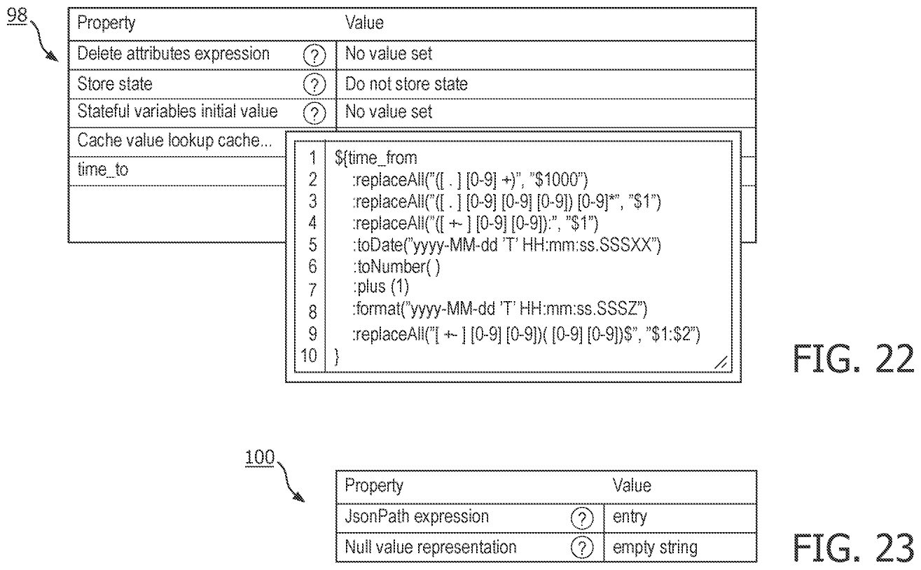

FIG. 19 is a schematic diagram that illustrates getting a number of entries (get count, FIG. 16A) retrieved from an entity tree 92, in accordance with an embodiment of the invention. This processor counts the number of records retrieved by the entity tree query.

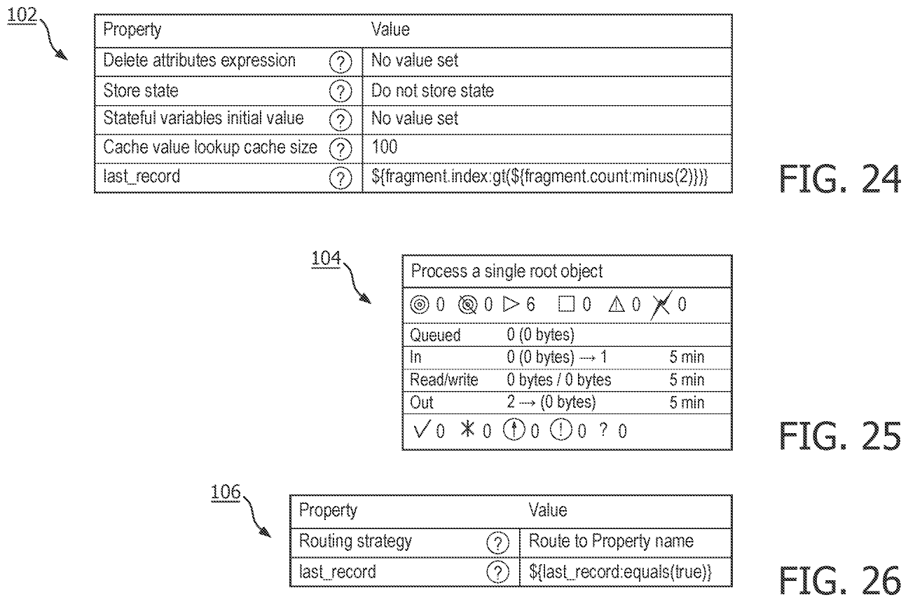

FIG. 20 is a schematic diagram that illustrates checking a number of entries as retrieved from an entity tree 94, in accordance with an embodiment of the invention. This processor checks the number of entries (e.g., presence of objects) that were retrieved from the entity tree using the specified max_record_count and time window. Depending on the result, the following actions are taken: (1) Count is zero: nothing was found in this time window. A split (e.g., splits a JSON File into multiple, separate FlowFiles for any array element) should not be attempted, since it will not output any FlowFile then, effectively stopping the pipeline. Therefore, the next time window should be retrieved (if appropriate); (2) Count is max: records were found in this time window, and there may be more. (There also might be exactly max_record_count items in this window, but this cannot be determined without querying for more). The items need to be processed by the split processor (see below), but first the end time is changed to one millisecond beyond the time stamp of the most recent record obtained now; (3) Count between zero and max: all records in this time window have been found. The end time can be kept as is and the objects can be routed to the split processor. In the split properties window, there is an assignment of the value to entry to split property called JsonPath Expression (where entry may be any specific JSON single root object required to Extra in ETL process).

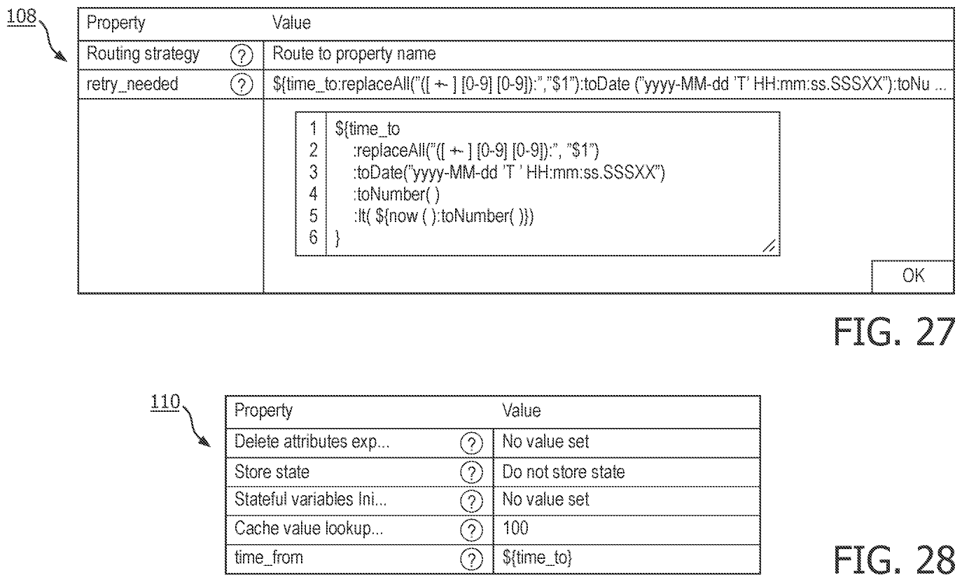

FIG. 21 is a schematic diagram that illustrates getting a time stamp of a last retrieved record (latest record time, FIG. 16B) 96, in accordance with an embodiment of the invention. This processor retrieves the last updated time stamp of the most recent record.

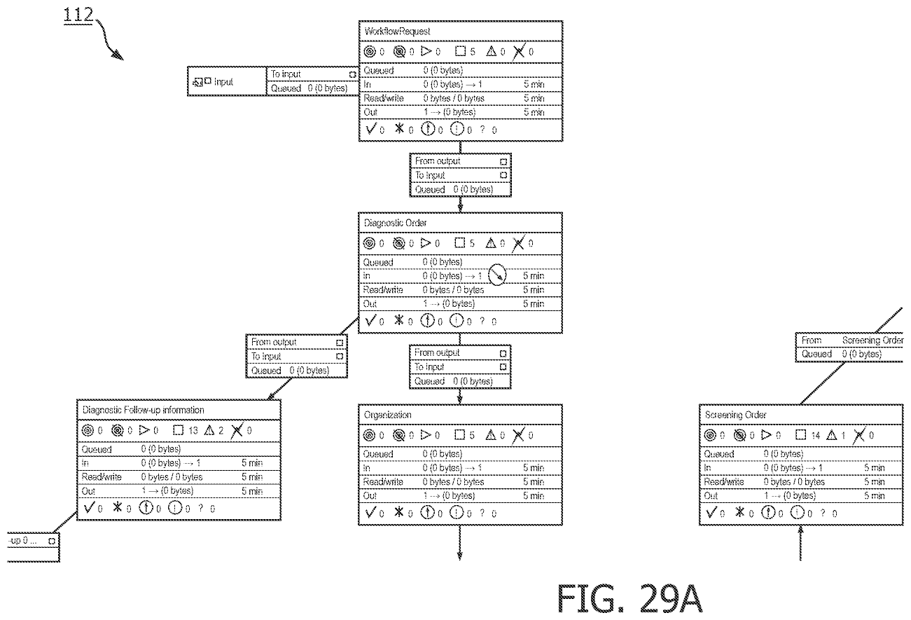

FIG. 22 is a schematic diagram that illustrates calculating a new end time (normalize end time, FIG. 16B) for a current time window if there are more records to be retrieved 98, in accordance with an embodiment of the invention. This processor sets the end time of the time window to the last updated time stamp of the most recent record, so that the next window starts from there and retrieves subsequent records. Note that in some embodiments, 1 millisecond is added to prevent the pipeline from coming in an infinite loop when there are max_record_count or more records with the same time stamp (which is trivially achieved if max_record_count is set to one).

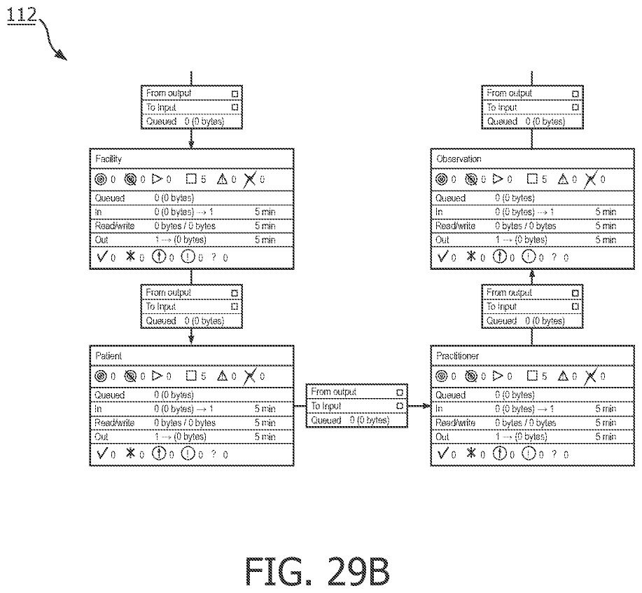

FIG. 23 is a schematic diagram that illustrates splitting an array of records into separate records (split root objects, FIG. 16B) 100, in accordance with an embodiment of the invention. This is a simple processor that splits the array of entries as retrieved in the query to the entity tree into separate items.

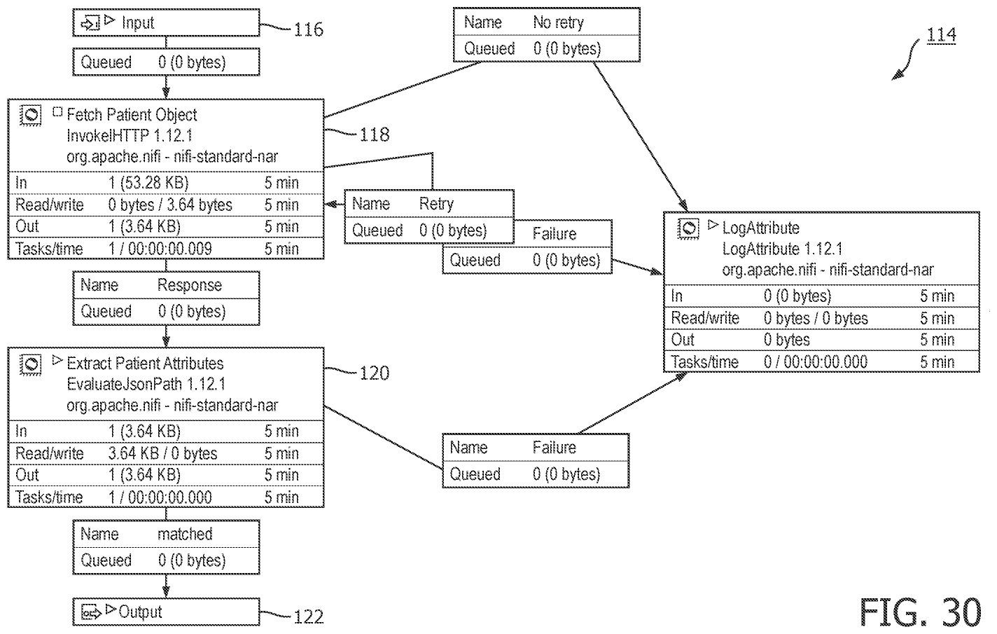

FIG. 24 is a schematic diagram that illustrates determining whether this is the last record of a split 102 (FIG. 16A), in accordance with an embodiment of the invention. This processor sets the last record flag on the last record of the split. This information is used further down the pipeline to trigger the next loop. Note that the fragment.index counts from 0 to fragment.count−1. The expression uses minus(2), as NiFi does not have an eq nor a le function.

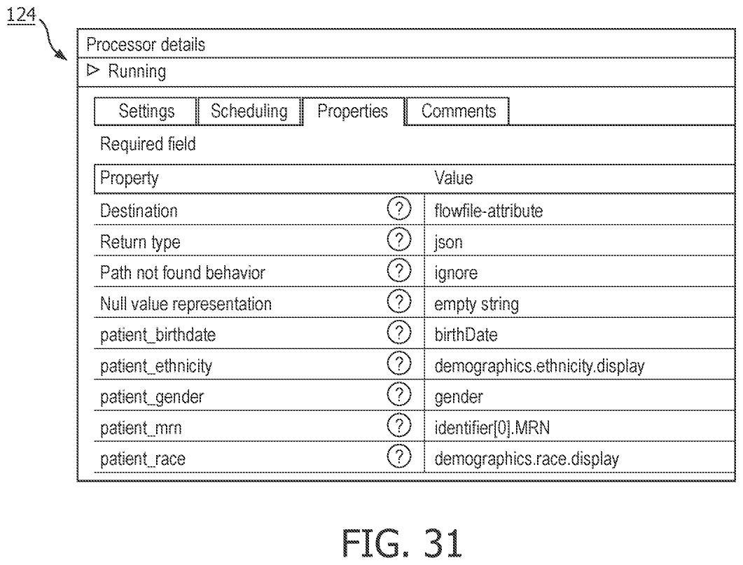

FIG. 25 is a schematic diagram that illustrates an example process group 104 (FIG. 16B) responsible for performing analytics application specific processing, in accordance with an embodiment of the invention. This processor takes a single entity tree object as content and performs all the functions necessary to insert a relevant record into the analytics database (e.g., specifies when and how the pipeline is triggered upon changes in the LCO workflows and events, such as based on experience, investigation, etc.). Note that this process group routes the FlowFile to the success output if it does not fail. This includes the cases where the entity tree object was correctly processed and inserted into the database or the entity tree object was deemed irrelevant (e.g., navigation was not completed yet).

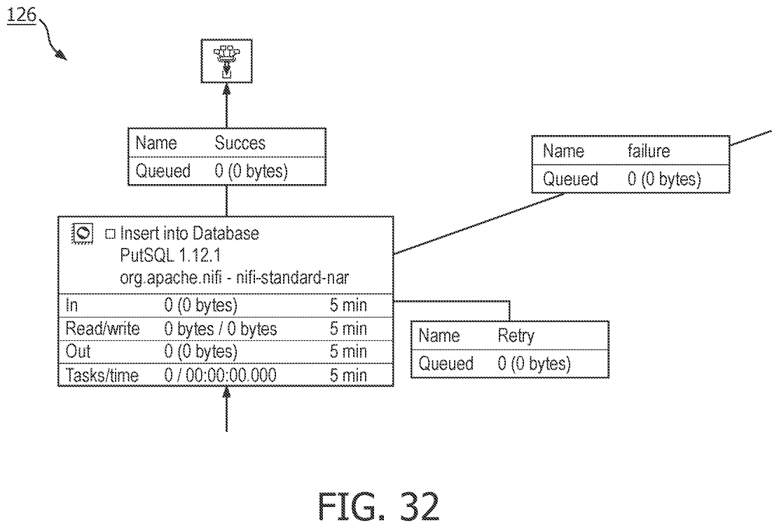

FIG. 26 is a schematic diagram that illustrates only triggering a next time fetch if the last record of the previous fetch is being processed 106, in accordance with an embodiment of the invention. This processor checks whether the record is the last record of the split. If so, the rest of the pipeline determines whether another fetch is needed. If not, the FlowFile is ignored (i.e., in the context of tracking the last record). While processing the multiple record called FlowFile in NiFi, each FlowFile is tracked using an attribute called last record, and the attribute value Boolean is updated, based on the record processed or not. This in turn facilitates fetching periodic records without disconnect from the flow till the last records on the present day are fetched (e.g., when executed by the reference of start date (historic date)).

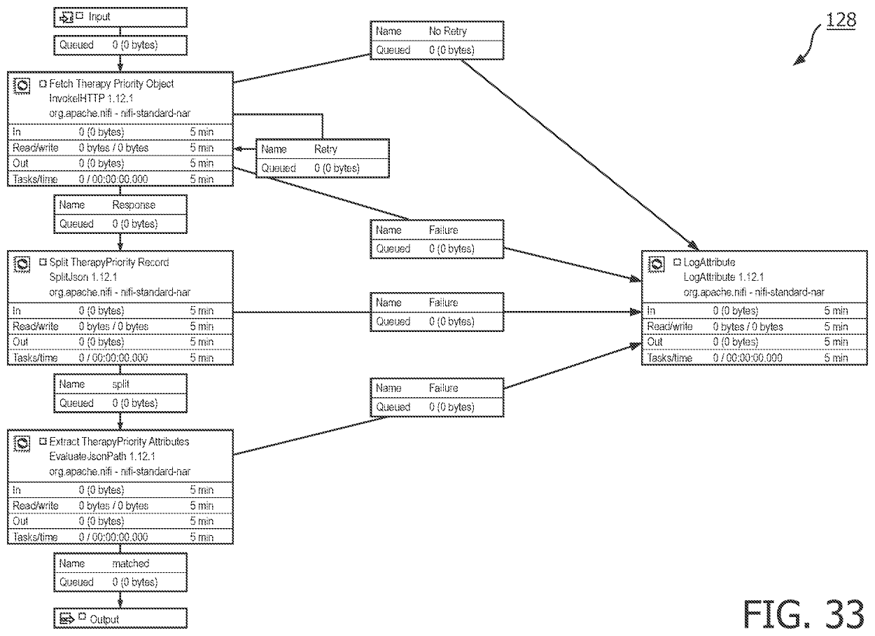

FIG. 27 is a schematic diagram that illustrates determining whether another fetch is needed (need to retry, FIG. 16A) 108, in accordance with an embodiment of the invention. This processor checks whether the current time window extends beyond now. If not, another fetch needs to be done. If so, this run can be successfully exited. Note how the same technique is used to interpret the end time as a string.

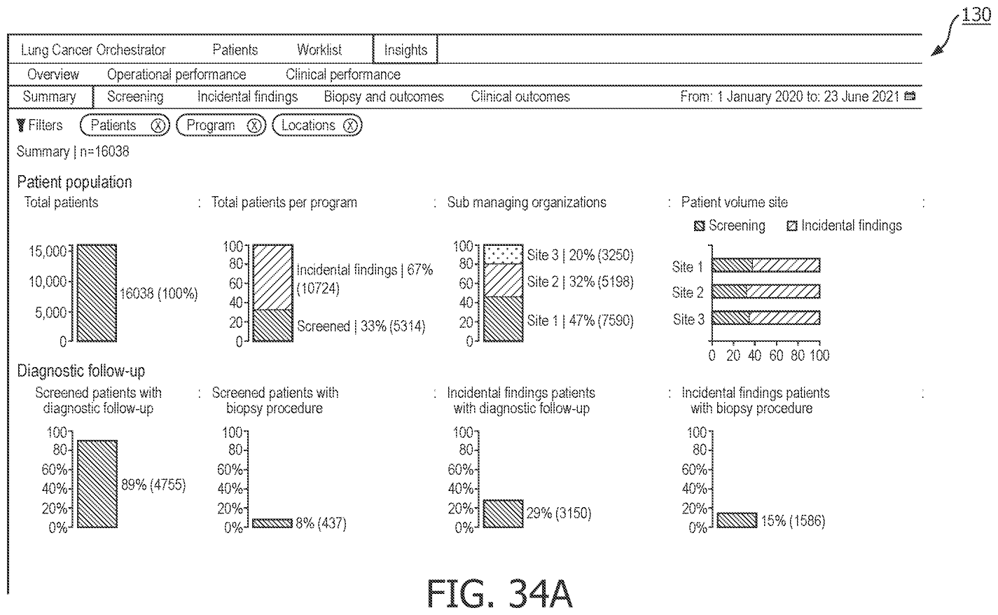

FIG. 28 is a schematic diagram that illustrates starting a new time window 110 (and see, also, FIG. 16A), in accordance with an embodiment of the invention. This processor sets the new start time to the old end time, to prepare for another fetch.

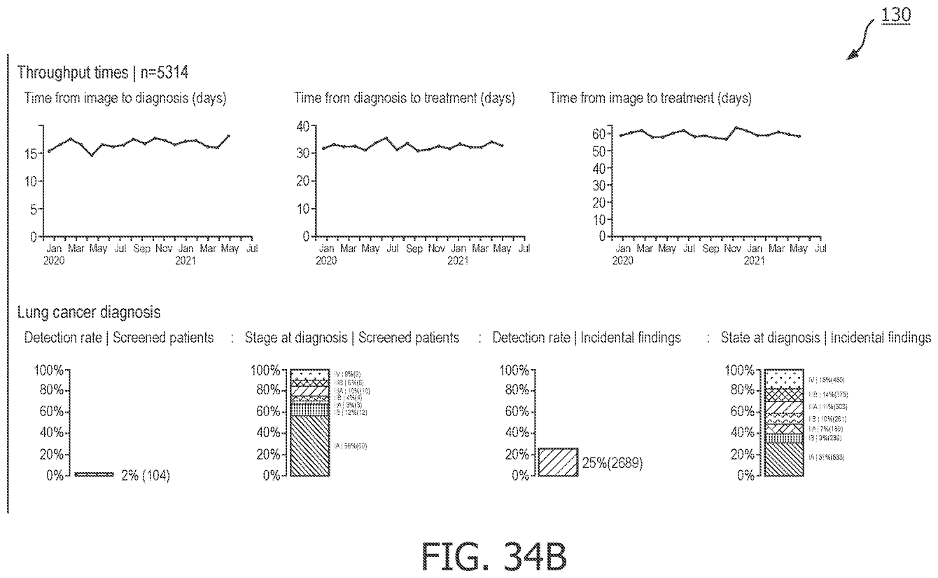

FIGS. 29A-29B are schematic diagrams that illustrate example process groups 112 for fetching entity tree objects, in accordance with an embodiment of the invention. For instance, FIGS. 29A-29B show how one NiFi process group is defined per object to be fetched from the entity tree. The root object is WorkflowRequest (described further below). From there, information for fetching the other objects is passed as FlowFile attributes. Each process group in FIGS. 29A-29B is also responsible for extracting information from the entity tree objects and storing them in FlowFile attributes.

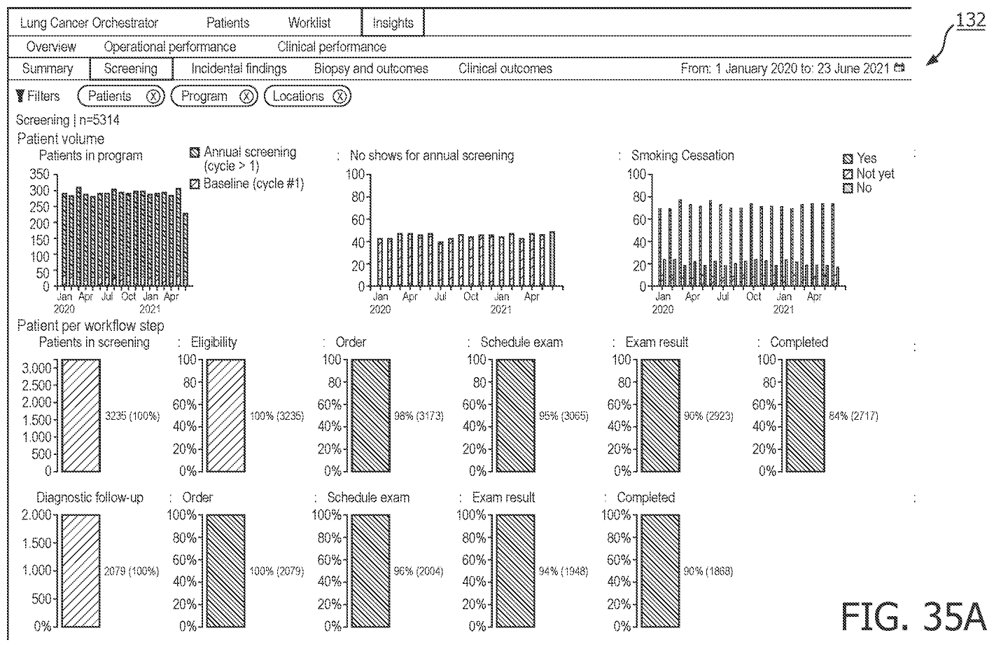

FIG. 30 is a schematic diagram that illustrates an example NiFi design pattern 114 for extracting and transforming information, in accordance with an embodiment of the invention. As would be appreciated by one having ordinary skill in the art, a NiFi user interface may be used to select (e.g., drag and drop) and configure the processor to what is displayed in the user interface. A large part of the information needed in the analytics table may be extracted directly from fields of the entity tree objects (sometimes in nested objects). The NiFi design pattern for this is shown in FIG. 30. In general, a process group for a particular object to be retrieved from the entity tree comprises an input named Input 116, a processor 118 to fetch the object and return the JSON-content, a processor 120 to copy data from the JSON content into FlowFile attributes, and an output named Output 122. The fetch patient object processor 118 retrieves the patient object from the entity tree. The extract patient attributes 120 fetches the relevant information from the patient object. The extracted information is stored in FlowFile attributes. These attributes have the same name as the corresponding columns of the analytics database.

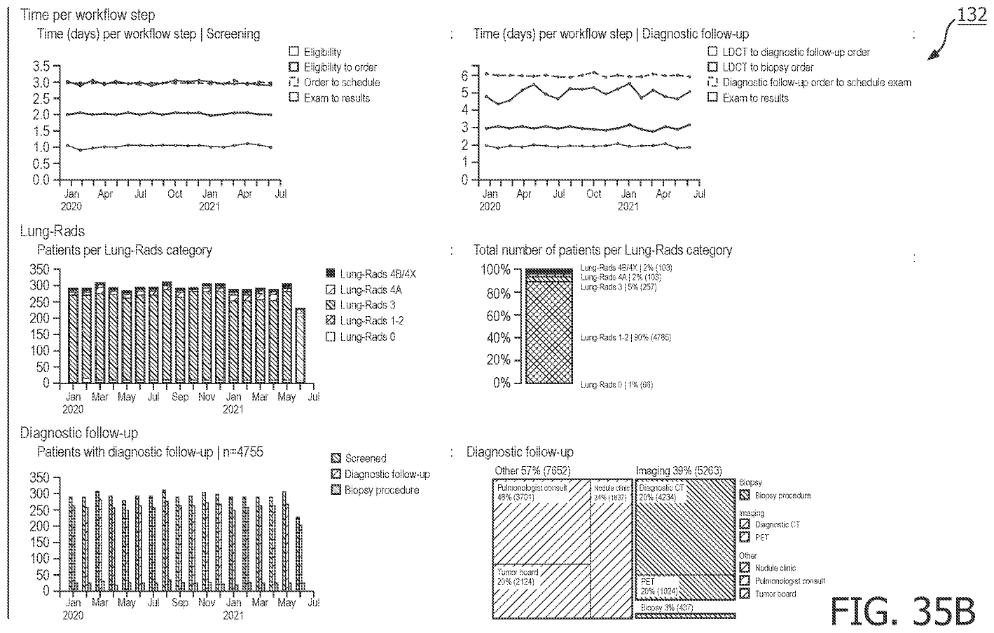

FIG. 31 is a schematic diagram that illustrates an example extraction and transformation 124 of patient attributes, in accordance with an embodiment of the invention.

The PUT SQL code fragment below shows how to insert a new record into the analytics database given information stored in FlowFile attributes. Note how the insert statement contains a list of database column names and a list of flow attributes from which the values are derived (usually but not always 1:1). These two lists should be kept in sync. The UPDATE part of the SQL statement contains the same information as the INSERT part, and should also be kept in sync.

INSERT INTO ${ database_schema }.${
screening_event_table_name }
(
logical_id,
last_updated,
organization_id,
screening_date,
screening_lung_rads_score,
screening_ct_examresult_modifier_S,
screening_ct_other_findings
...
)
VALUES
(
‘${workflow_request_id}’,
‘${last_updated}’:: timestamp WITH time zone,
‘${organization_id}’,
(CASE WHEN ‘${screening_date}’ IN (‘’) THEN NULL ELSE
‘${screening_date}’ end)::
timestamp WITH time zone,
‘${screening_lung_rads_score}’,
‘${screening_ct_examresult_modifier_S}’,
‘${screening_ct_other_findings}’
...
)
ON CONFLICT( logical_id )DO UPDATE SET
(
logical_id,
last_updated,
organization_id,
screening_date,
screening_lung_rads_score,
screening_ct_examresult_modifier_S,
screening_ct_other_findings
...
)
=
(
‘${workflow_request_id}’,
‘${last_updated}’:: timestamp WITH time zone,
‘${organization_id}’,
(CASE WHEN ‘${screening_date}’ IN (‘’) THEN NULL ELSE
‘${screening_date}’ end)::
timestamp WITH time zone,
‘${screening_lung_rads_score}’,
‘${screening_ct_examresult_modifier_S}’,
‘${screening_ct_other_findings}’
...
)

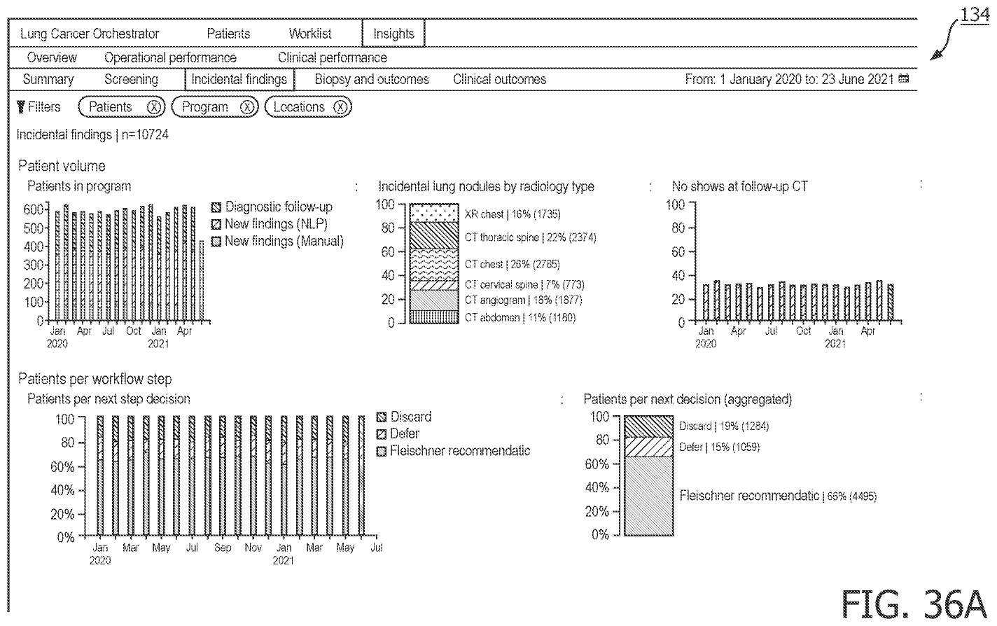

FIG. 32 is a schematic diagram that illustrates putting data into an analytics database 126, in accordance with an embodiment of the invention. For instance, FIG. 32 shows the NiFi processor with the INSERT statement. Currently, log attributes containing log level information, error, and warn are captured as features.

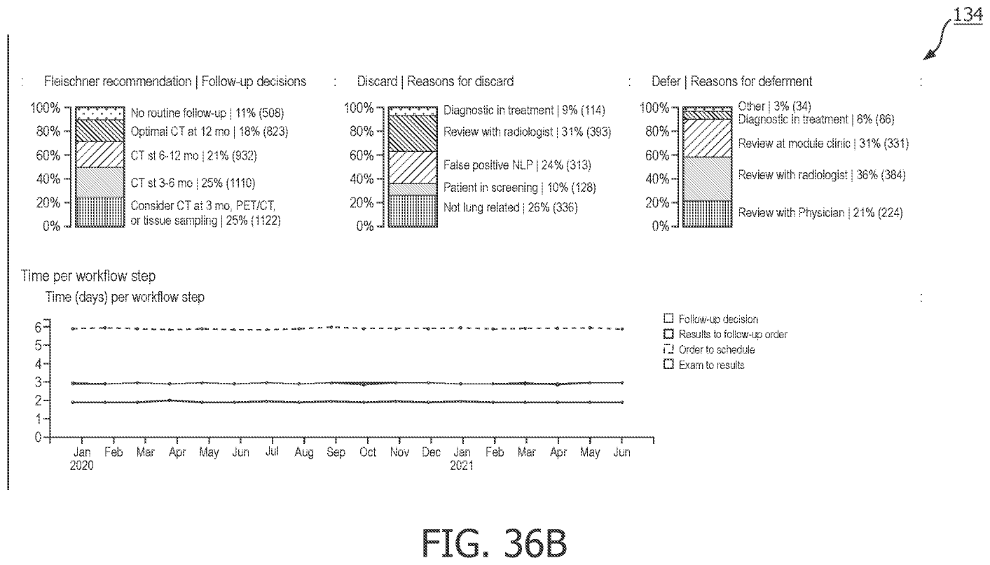

FIG. 33 is a schematic diagram that illustrates an example of detailed information 128 of each processor inside a process group, in accordance with an embodiment of the invention. FIG. 33 illustrates a way to capture error in the pipeline during ETL. Referring to FIG. 33 as reading from left to right, all the processors are connecting to the log attribute processor on failure, which means any left processor failure message is tracked, and while doing so, only related insensitive attributes information is captured/filtered. A log attribute processor handles the error across the process group. In one embodiment, during capturing of logs, only information is captured that is not clinically sensitive. The table below contains the fields that are ignored while capturing the logs (for logging purposes, clinically sensitive information is filtered out for the above-described database tables).

| | | | |
|---|---|---|---|
| workflow_request_id | workflow_stopped | workflow_step | workflow_revision_id |
| time_to | time_from | facility_id | organization_id |
| max_record_count | window_size | | |

Attention is now directed to visualization of the data contained in the databases. The data from the analytics database may be loaded in either a custom built or integrated analytics application. In the example below, and in one embodiment, a business intelligence application from an external party is used to visualize the extracted data from the lung cancer orchestrator, and has built-in features to connect to several types of databases and plot intuitive visuals and charts. In the description that follows, a general setup of lung analytics dashboards is disclosed. Note that in some embodiments, other visualization platforms may be used. The following setup is considered to be generalizable to other visualization platforms.

Data sources are defined that specify the database connections used by the visualization platform. These may comprise the following, beginning with database connections:

| Item | Description |
|---|---|
| Connection | Connection to the data sources |
| Tables | Select the Lung table or write a custom SQL query to generate the dataset. We |
| | connect to ‘lung_screening_events’, |
| | ‘lung_diagnostic_followup_events’, tables containing Lung screening workflow, |
| | as well as ‘lung_incidents’ for the incidental findings workflow. |
| Fields | Define data columns as attributes, dates, integers and user-facing names for |
| | each column. Create custom and derived metrics |
| Refresh | Scheduled periodic refreshing of metadata and clearing of cache on an hourly |
| | basis. |
| Visuals | Select the kind of visuals that would be supported by the dashboard. |

Subsequently a mapping is created of database column names to chart names:

| Column Name | Chart name |
|---|---|
| logical_id | Logical Id |
| last_updated | Last Updated |
| organization_id | Organization Id |
| facility_id | Facility Id |
| etl_job_id | Etl Job Id |
| etl_date | Etl Date |
| content jsonb | Content |
| workflow_request_id | Workflow Request Id |
| workflow_revision_id | Workflow Revision Id |
| order_category_code | Order Category Code |
| order_category_display | Order Category Display |
| event_category_code | Event Category Code |
| event_category_display | Event Category Display |
| event_group_code | Event Group Code |
| event_group_display | Event Group Display |
| pathology_event_technique_code | Pathology Event Technique Code |
| pathology_event_technique_display | Pathology Event Technique Display |

The below table is a lung_screening_events table:

| Column Name | Chart name |
|---|---|
| logical_id | Logical Id |
| last_updated | Last Updated |
| organization_id | Organization Id |
| facility_id | Facility Id |
| etl_job_id | Etl Job Id |
| etl_date | Etl Date |
| content jsonb | Content |
| workflow_revision_id | Workflow Revision Id |
| workflow_step | Workflow Step |
| workflow_stopped | Workflow Stopped |
| workflow_stopped_reason | Workflow Stopped Reason |
| observation_smoking_cessation | Observation Smoking Cessation |
| organization_name | Organization Name |
| facility_name | Facility Name |
| practitioner_id | Practitioner Id |
| practitioner_name | Practitioner Name |
| patient_mrn | Patient Mrn |
| patient_id | Patient Id |
| event_id | Event Id |
| order_category_code | Order Category Code |
| order_category_display | Order Category Display |
| event_category_code | Event Category Code |
| event_category_display | Event Category Display |
| event_group_code | Event Group Code |
| event_group_display | Event Group Display |
| screening_date | Screening Date |
| screening_lung_rads_score | Screening Lung Rads Score |
| screening_ct_other_findings | Screening ct Other Findings |
| screening_ct_examresult_modifier_S | Screening Ct Examresult Modifier S |

A similar mapping is made for the incidental findings analytics data source configuration.

As to volume, custom, and/or derived fields, subsequently, custom and derived metrics may be defined. These are metrics that may be created using built-in data processing editors available in the used visualization platform, supporting SQL-like operations. The ‘Volume’ metric used in all the dashboards is automatically calculated and named as ‘Number of Cycles’. The following Derived Field are created for lung analytics dashboards:

| Field | Chart Name | Query |
|---|---|---|
| Derived Field | Pathology | CASE |
| | | WHEN event_category_code = ‘pathology-lung’ |
| | | THEN pathology_event_technique_display |
| | | ELSE ‘NA’ |
| | | END |
| Derived Field | Others | CASE |
| | | when event_category_code = ‘molecularTesting’ |
| | | then ‘Molecular testing’ |
| | | when event_category_code = ‘specialist-consult’ |
| | | then ‘Specialist consult’ |
| | | when event_category_code = ‘pnc’ then |
| | | ‘Pulmonary Nodule Clinic’ |
| | | when event_category_code = ‘other’ then ‘Other’ |
| | | ELSE ‘NA’ |
| | | END |
| Derived Field | Imaging | CASE |
| | | when event_category_code = ‘pet-ct-default’ then |
| | | ‘PET-CT’ |
| | | when event_category_code = ‘Idct’ then ‘Screening |
| | | CT-Lung’ |
| | | when event_category_code = ‘ct-lung’ then ‘Chest |
| | | CT’ |
| | | ELSE ‘NA’ |
| | | END |
| Derived Field | Number of | CASE WHEN screening_date=first_screening_date |
| | Visits | THEN ‘Baseline’ ELSE ‘Annual Cycle’ END |
| Derived Field | Lung Rads | CASE |
| | Score Category | WHEN screening_lung_rads_score_code = ‘0’ |
| | | THEN ‘Lung-RADS 0’ |
| | | WHEN screening_lung_rads_score_code = ‘1’ |
| | | THEN ‘Lung-RADS 1’ |
| | | WHEN screening_lung_rads_score_code =‘2’ |
| | | THEN ‘Lung-RADS 2’ |
| | | WHEN screening_lung_rads_score_code = ‘3’ |
| | | THEN ‘Lung-RADS 3’ |
| | | WHEN screening_lung_rads_score_code = ‘4A’ |
| | | THEN ‘Lung-RADS 4A’ |
| | | WHEN screening_lung_rads_score_code = ‘4B’ |
| | | THEN ‘Lung-RADS 4B’ |
| | | WHEN screening_lung_rads_score_code = ‘4X’ |
| | | THEN ‘Lung-RADS 4X’ |
| | | ELSE ‘NA’ |
| | | END |
| Derived Field | Lung-RADS | CASE |
| | modifier S | WHEN |
| | | screening_ct_examresult_modifier_s_code=‘Y’ THEN |
| | | ‘Yes’ |
| | | WHEN |
| | | screening_ct_examresult_modifier_s_code=‘N’ |
| | | THEN ‘No’ |
| | | ELSE ‘Not Specified’ |
| | | END |
| Custom Metric | Diagnostic | SUM (CASE WHEN workflow_step = |
| | follow-up | ‘UIDiagnosticFollowupCompleted’ or |
| | | (workflow_step=‘diagnosticFollowUp’) THEN 1 ELSE |
| | | 0 END) |
| Custom Metric | Screened | COUNT (screening_date) |

A variety of analytics dashboards are made, comprising, but not limited to: summary (e.g., high level summary overview of all key analytical insights), lung cancer screening (e.g., screening volumes, Lung-RADS scores, other findings, diagnostic follow-up decisions, breakdown of diagnostic follow-up events), incidental findings (e.g., volume of new findings, follow-up decisions, breakdown of the follow-up decisions (e.g. Fleischner recommendations), diagnostic follow-up decisions, breakdown of diagnostic follow-up events), biopsy and outcomes (e.g., tissue sampling procedures, outcomes from the tissue sampling procedures, tissue diagnoses and diagnoses per tissue sampling procedure type and lung cancer and other cancer detection rate), and clinical outcomes (e.g., volume of lung cancer detected at stage I&II, stage distribution, cell types and molecular profiles, time to diagnosis and time to treatment, volume of given treatments and breakdown per patient demographics).

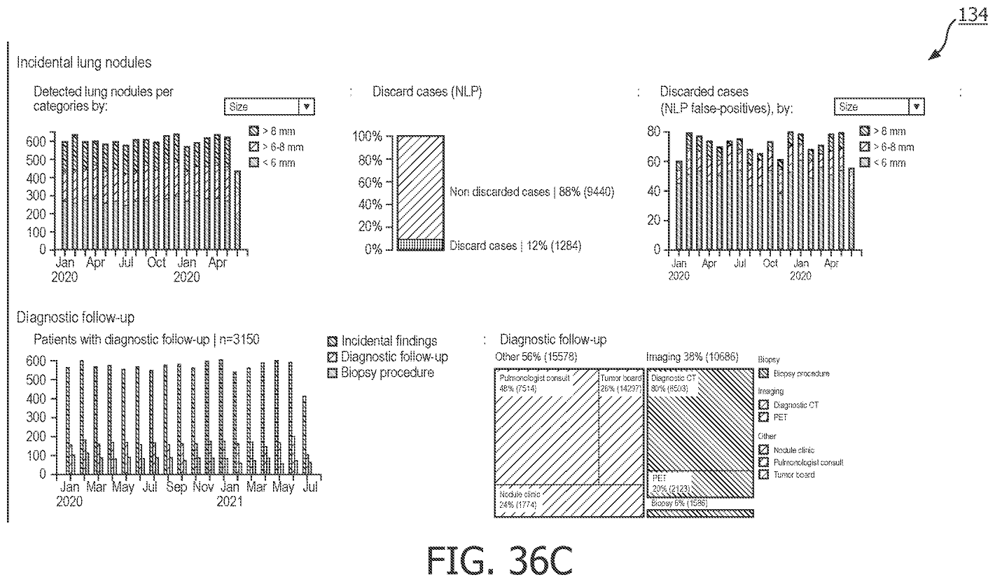

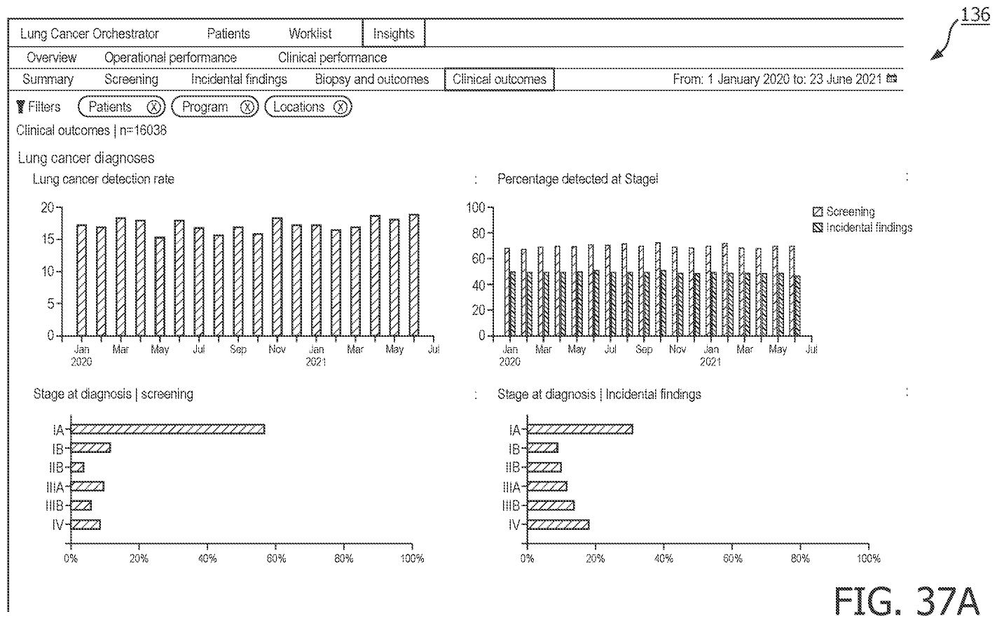

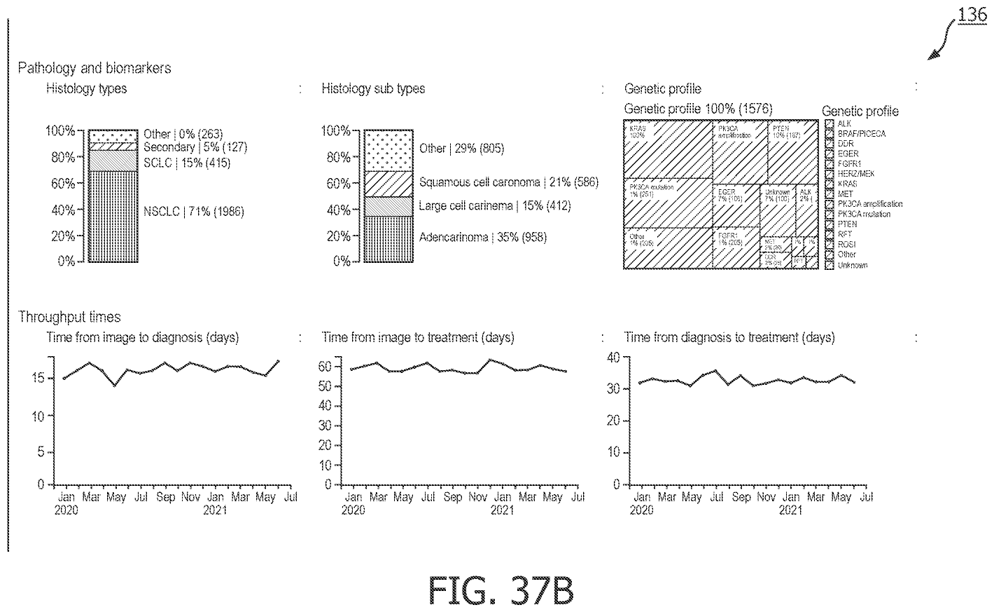

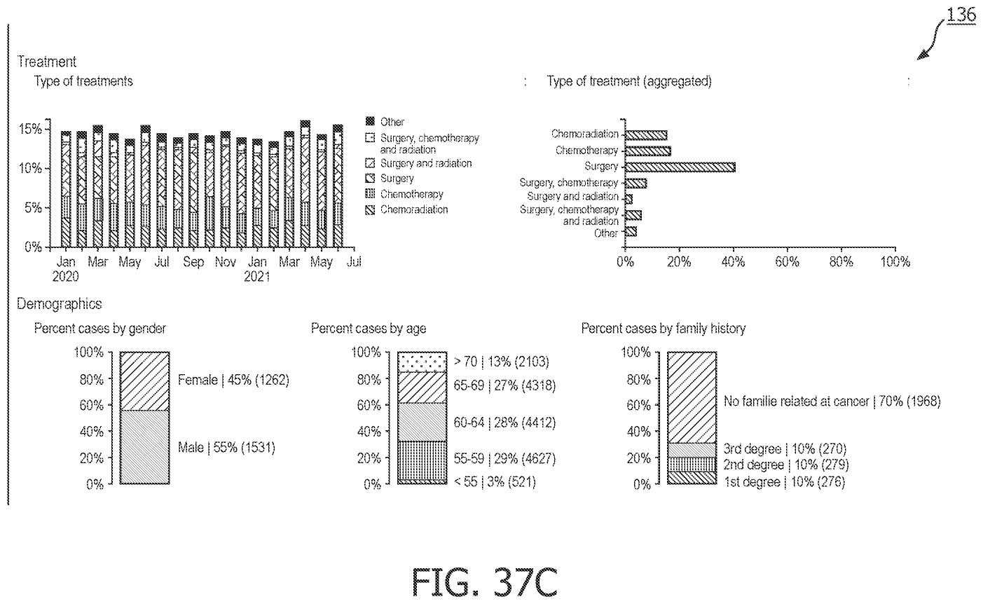

The dashboards may be filtered by a specific time period, in which the data displayed on the dashboard is filtered and binned by the date of the procedures and date of the decisions made in the patient management application. The dashboards may be filtered by facility to show data for one specific hospital facility, or show data of multiple facilities. Some example dashboards are depicted in FIGS. 34A-37C, and include a lung analytics summary dashboard 130 (FIGS. 34A-34B), lung analytics screening dashboard 132 (FIGS. 35A-35B), lung analytics biopsy and outcomes dashboard 134 (FIGS. 36A-36C), and lung analytics clinical outcomes dashboard 136 (FIGS. 37A-37C).

In view of the above description, it should be appreciated that several areas of improvement over the state of the art include the LCO-ETL pipeline connections (e.g., how the pipelines are connected to and triggered by selected workflow changes and data as captured in the entity tree objects), dynamic fetching and scalability, and cross care continuum and cross domain analytics (e.g., solutions working in cohesion to provide unique insights that could otherwise not be extracted). Improvements in the state of the art include the way the data structures are constructed and the way the ETLs are designed and configured and connected to the integrated lung nodule management application. Relating to the above description, innovations are found in several aspects, including (1) how the database tables are derived and constructed from the lung cancer orchestrator described in FIG. 2, (2) how the ETL described above is connected to the lung cancer orchestrator application and triggered to incrementally load data upon specific workflow changes in the application, and (3) a recognition that the analytics application does not simply ingest data directly coming out of the LCO and store the same in a database, but rather, that certain embodiments of an analytics application derives these analytical insights through a combination of: monitoring specific workflow statuses, specific data points captured in these workflows and derive metrics from multiple of these data points.

Explaining further with illustrations, with regard to the LCO-ETL connections, the disclosed embodiments illustrate an analytics application utilizing ETL pipelines connected to workflows from an integrated lung nodule management application (covering both lung cancer screening and incidental findings management) and transforming data captured during execution of the workflows into key performance indicators (KPIs). How the NiFi pipelines are designed and setup, as explained above, to extract information in an incremental way from a lung nodule patient management application, that not only covers screening, but also incidental findings, and also the multidisciplinary decision-making workflows after that, are all improvements to the state of the art. In effect, the analytics application is able to relate the very initial nodule finding to all subsequent follow-up steps and diagnoses.

The pipelines observe workflows and incrementally load the data into the analytics database, which enables real-time or near-real-time monitoring of the nodule management workflows and bottlenecks in the workflows. This is in contrast to providing a monthly report, or reporting for only a subset of metrics. The pipelines are specific in only fetching the relevant data to derive KPIs from the lung nodule management application, such as patient volumes, patients per workflow step or follow-up decision, breakdown per Lung-RADS (screening) or Fleischner (Incidental findings) category, additional diagnostic testing performed, biopsy results and lung cancer detection rates. The data may cover clinical, operational, economic and staffing aspects.

Note that for the analytics, information may be derived from the data in the entity tree (i.e., it is not only a 1-1 display into the analytics application). Derivation is often a combination of a data point with a workflow status, or a derivative of 2 data points. For instance, from observing the existence of 2 screening exams with 2 different dates, derivation includes a determination of which is the baseline exam and which is the follow-up screening exam. Fetches are based on changes in the workflows that trigger the pipeline, and which are only counted when the workflows status is completed. As another example, through extraction of the time at which exams were ordered, scheduled and reviewed (having exam results), throughput times may be derived. By retrieval of data from when the report was generated of different types of diagnostic events (e.g. imaging and pathology), the exact time from image to tissue diagnosis may be derived. As an additional illustration, from observing both the Lung-RADS score (radiological risk score) from an exam, the follow-up decisions taken in the application, and if a tissue sampling was done, various computations may be performed (e.g., tissue sampling rate per Lung-RADS category, etc.). As yet another example, cancer detection rate may be derived through count of all screening exams versus the exams results that have at least 1 diagnostic follow-up event with a lung cancer tissue diagnosis, derived from the tissue diagnosis type entered in the application.

Another beneficial result possible from the LCO-ETL connections involves the detection of bottle necks and non-compliance. For instance, by applying upper- and lower limits on KPIs related to these workflows (e.g., time to diagnosis), the pipelines may detect if workflows start running out of time and can generate an alert. As another example, through monitoring follow-up decisions in relation to detected nodules and the characteristics of the nodules, the analytics application timely reflects if follow-up decisions are being taken in a non-compliant way (as these findings are managed based on, for instance, international guidelines). As a further illustration, detection of bottlenecks or non-compliance in the workflows of a cohort of patients may aid in triggering interventions at personnel level (e.g., through monitoring of volume of exams ordered and reviewed, time between order and review and total number of logged in users). Also, the type of exam that triggers the highest number of incidental findings may be identified, which can be further analyzed to see if findings identified from particular exam types result in further diagnostic follow-up and appear to be cancer more frequently than of others.

As to dynamic fetching and scalability, the pipelines dynamically fetch value sets from configured workflows in the patient management applications, which enables scaling to other disease areas for screening of other cancer types or management of other incidental findings (e.g., change of the configuration of the major workflow steps and value sets in the patient management application may provide a ‘new’ analytics application).

With respect to the cross care continuum features, the lung cancer orchestrator, pulmonary nodule clinic and multidisciplinary team orchestrator are applications that span the lung cancer care continuum and are all implemented, in one embodiment, on the same cloud platform (e.g., IntelliSpace Precision Medicine). This platform also comprises an application to interpret genetic data (Genomics workspace) and that captures treatment decisions (Oncology Pathways application). All data from these applications are stored in the entity tree. By joining data from the entity tree, KPIs may be derived from combining data that are normally scattered across applications. Augmenting these analytical insights with data from the computer-aided nodule detection and characterization application (e.g., DynaCAD) and patient engagement application enables extracting insights from solutions working in cohesion [e.g., commonalities in diagnostic delays (e.g. patients with multiple reported comorbidities, typically the following diagnostic tests were forgotten, typically these were the smaller nodules that required more discussion time and testing), and/or commonalities in genomic profile of found cancers].

With respect to combining data from various sources, also data from legacy platforms may be combined into new platforms (e.g., expanding the data, including prior data, etc.), including, for instance, data from on premise to cloud platforms, data with different data base structures, etc. Analysis of the potential impact of updating/changing patient management workflows on nodule management program efficacy and downstream revenue through simulating workflows is also enabled. Also, natural language processing (NLP) algorithm findings in radiology reports in relation to follow-up decisions may be used, providing real-world evidence of NLP performance.

Though various embodiments have been disclosed, it should be appreciated by one having ordinary skill in the art, in the context of the present disclosure, that other embodiments are also contemplated. For instance, in one embodiment, time intervals of data extraction may be configured according to user preferences. There is an opportunity to configure the pipelines to extract data at a close to real-time (e.g., hourly) basis, enabling users to see in real-time or near real-time the impact of their actions taken in the patient management application on the metrics displayed in the analytics application. This could also aid in bottleneck identification. In some embodiments, workflows in the lung cancer orchestrator or ISPM platform may be configured to accommodate alternative workflows or value sets, for lung nodule management of management of other findings. In some embodiments, the analytics application's ETL pipelines and dashboards may be configured to dynamically fetch data from alternative workflows or value sets. In some embodiments, there may be expansion of the ETLs to extract data from other workflow management applications, imaging applications or hospital information management system and combine the insights with the information extracted from the ISPM workflows. In some embodiments, staff productivity may be derived from volumes of exams reviewed by unique users of the patient management application. In some embodiments, revenue may be derived from volume of exams and volume of follow-up procedures and specification of procedure cost and reimbursement and staff cost.

In view of the above disclosure, one having ordinary skill in the art would appreciate that one embodiment of a method is disclosed that is performed by a computing device executing an analytics application used in conjunction with a patient management application, the method comprising: receiving workflows and events from the patient management application, the workflows and events corresponding to patient data; selectively processing the workflows and events in extract, transform, and load (ETL) pipelines responsive to trigger points in the workflows; loading, by the ETL pipelines, data resulting from the selective processing into a data analytics data structure used to enable visualization of patient data and derived metrics or key performance indicators.

Note that the analytics application (e.g., as depicted in FIG. 1), and the patient management application within which the analytics application is embedded, may be implemented as part of a cloud computing environment (or other server network) that serves one or more clinical and/or research facilities. When implemented as part of a cloud service or services, one or more computing devices may comprise an internal cloud, an external cloud, a private cloud, or a public cloud (e.g., commercial cloud). For instance, a private cloud may be implemented using a variety of cloud systems including, for example, Eucalyptus Systems, VMWare vSphere®, or Microsoft® HyperV. A public cloud may include, for example, Amazon EC2®, Amazon Web Services®, Terremark®, Savvis®, or GoGrid®. Cloud-computing resources provided by these clouds may include, for example, storage resources (e.g., Storage Area Network (SAN), Network File System (NFS), and Amazon S3®), network resources (e.g., firewall, load-balancer, and proxy server), internal private resources, external private resources, secure public resources, infrastructure-as-a-services (IaaSs), platform-as-a-services (PaaSs), or software-as-a-services (SaaSs). The cloud architecture of the computing devices may be embodied according to one of a plurality of different configurations. For instance, if configured according to MICROSOFT AZURE™, roles are provided, which are discrete scalable components built with managed code. Worker roles are for generalized development, and may perform background processing for a web role. Web roles provide a web server and listen for and respond to web requests via an HTTP (hypertext transfer protocol) or HTTPS (HTTP secure) endpoint. VM roles are instantiated according to tenant defined configurations (e.g., resources, guest operating system). Operating system and VM updates are managed by the cloud. A web role and a worker role run in a VM role, which is a virtual machine under the control of the tenant. Storage and SQL services are available to be used by the roles. As with other clouds, the hardware and software environment or platform, including scaling, load balancing, etc., are handled by the cloud.

In some embodiments, the computing devices may be configured into multiple, logically-grouped servers (run on server devices), referred to as a server farm. The computing devices may be geographically dispersed, administered as a single entity, or distributed among a plurality of server farms. The computing devices within each farm may be heterogeneous. One or more of the computing devices may operate according to one type of operating system platform (e.g., WINDOWS NT, manufactured by Microsoft Corp. of Redmond, Wash.), while one or more of the computing devices may operate according to another type of operating system platform (e.g., Unix or Linux). The computing devices may be logically grouped as a farm that may be interconnected using a wide-area network (WAN) connection or medium-area network (MAN) connection. The computing devices may each be referred to as, and operate according to, a file server device, application server device, web server device, proxy server device, or gateway server device.

Note that cooperation between devices (e.g., clinician computing devices) of other networks and the devices of the cloud (and/or cooperation among devices of the cloud) may be facilitated (or enabled) through the use of one or more application programming interfaces (APIs) that may define one or more parameters that are passed between a calling application and other software code such as an operating system, library routine, and/or function that provides a service, that provides data, or that performs an operation or a computation. The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer employs to access functions supporting the API. In some implementations, an API call may report to an application the capabilities of a device running the application, including input capability, output capability, processing capability, power capability, and communications capability.

As should be appreciated by one having ordinary skill in the art, one or more computing devices of the cloud platform (or other platform types), as well as of other networks communicating with the cloud platform, may be embodied as an application server, computer, among other computing devices. In that respect, one or more of the computing devices comprises one or more processors, input/output (I/O) interface(s), one or more user interfaces (UI), which may include one or more of a keyboard, mouse, microphone, speaker, tactile device (e.g., comprising a vibratory motor), touch screen displays, etc., and memory, all coupled to one or more data busses.

The memory may include any one or a combination of volatile memory elements (e.g., random-access memory RAM, such as DRAM, and SRAM, etc.) and nonvolatile memory elements (e.g., ROM, Flash, solid state, EPROM, EEPROM, hard drive, tape, CDROM, etc.). The memory may store a native operating system, one or more native applications, emulation systems, or emulated applications for any of a variety of operating systems and/or emulated hardware platforms, emulated operating systems, etc. In some embodiments, a separate storage device may be coupled to the data bus or as a network-connected device. The storage device may be embodied as persistent memory (e.g., optical, magnetic, and/or semiconductor memory and associated drives). The memory comprises an operating system (OS) and application software, including the analytics application described herein.

Execution of the software may be implemented by one or more processors under the management and/or control of the operating system. The processor may be embodied as a custom-made or commercially available processor, a central processing unit (CPU) or an auxiliary processor among several processors, a semiconductor based microprocessor (in the form of a microchip), a macroprocessor, one or more application specific integrated circuits (ASICs), a plurality of suitably configured digital logic gates, and/or other well-known electrical configurations comprising discrete elements both individually and in various combinations to coordinate the overall operation of the computing device.

When certain embodiments of the computing device are implemented at least in part with software (including firmware), it should be noted that the software may be stored on a variety of non-transitory computer-readable (storage) medium for use by, or in connection with, a variety of computer-related systems or methods. In the context of this document, a computer-readable storage medium may comprise an electronic, magnetic, optical, or other physical device or apparatus that may contain or store a computer program (e.g., executable code or instructions) for use by or in connection with a computer-related system or method. The software may be embedded in a variety of computer-readable storage mediums for use by, or in connection with, an instruction execution system, apparatus, or device, such as a computer-based system, processor-containing system, or other system that can fetch the instructions from the instruction execution system, apparatus, or device and execute the instructions.

When certain embodiments of the computing device are implemented at least in part with hardware, such functionality may be implemented with any or a combination of the following technologies, which are all well-known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), relays, contactors, etc.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

Note that various combinations of the disclosed embodiments may be used, and hence reference to an embodiment or one embodiment is not meant to exclude features from that embodiment from use with features from other embodiments. In the claims, the word “comprising” does not exclude other elements or steps, and the indefinite article “a” or “an” does not exclude a plurality. Further, each method claim may be performed by a computing device, system, or by a non-transitory computer readable medium. The computing device may include memory in the form of a non-transitory computer readable medium, or may include one or more each of a memory and a non-transitory computer readable medium. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms.

1. A method performed by a computing device executing an analytics application used in conjunction with a patient management application, the method comprising:
receiving workflows and events from the patient management application, the workflows and events corresponding to patient data;
selectively processing the workflows and events in extract, transform, and load (ETL) pipelines responsive to trigger points in the workflows; and
loading, by the ETL pipelines, data resulting from the selective processing into a data analytics data structure used to enable visualization of patient data and derived metrics or key performance indicators.

2. The method of claim 1, wherein the patient management application comprises a lung nodule management application, and the analytics application comprises a lung analytics application.

3. The method of claim 1, wherein the lung nodule management application manages the patient data for lung cancer screening and pulmonary incidental findings.

4. The method of claim 1, wherein the selective processing comprises transforming select patient data relevant to monitoring and/or a patient or cohorts of patients based on the lung cancer screening and the incidental pulmonary findings.

5. The method of claim 1, wherein the selective processing comprises transforming select patient data into metrics or key performance indicators.

6. The method of claim 1, wherein the ETL pipelines are configured to constrain fetching of the patient data in the workflows to patient data relevant to deriving the key performance indicators from the patient management application.

7. The method of claim 1, wherein the relevant patient data corresponds to one or more of clinical, operational, economic, or staffing functions in an organization.

8. The method of claim 1, wherein the relevant patient data corresponds to one or more of the following: patient volumes, patients per workflow step or follow-up decision, breakdown per Lung-RADS (screening) or Fleischner (Incidental findings) category, additional diagnostic testing performed, biopsy results, lung cancer detection rates, stage information and throughput times.

9. The method of claim 1, wherein the data analytics data structure enables one or more of real-time monitoring, or near real-time monitoring, of the workflows for bottlenecks or non-compliance in the workflows.

10. The method of claim 1, wherein the monitoring for the bottlenecks further comprises applying limits on the key performance indicators that enable a trigger by the ETL pipelines when the workflows exceed the limits, and wherein the monitoring for the non-compliance comprises monitoring the workflows of a cohort of patients.

11. The method of claim 1, further comprising providing an alert when the workflows exceed the limits or triggering interventions at a personnel level based on the non-compliance.

12. The method of claim 1, wherein receiving the workflows and events data comprises receiving the workflows via an entity tree.

13. The method of claim 1, wherein selectively processing the workflows comprises deriving information from the entity tree, the deriving comprising one or more of a combination of a data point with a workflow status or a derivative from two or more data points.

14. The method of claim 1, wherein selectively processing the workflows further comprises monitoring follow-up decisions in relation to detection of suspected disease, the monitoring further comprising determining if follow-up decisions are being taken in a non-compliant manner.

15. The method of claim 1, wherein the selectively processing of the workflows further comprises dynamically fetching value sets from the workflows, the dynamic fetching enabling application to other diseases or management of other types of incidental findings.

16. The method of claim 1, wherein the patient management application comprises one or more of the following implemented in a cloud computing service: lung cancer orchestrator, comprising a computer aided detection module, lung cancer screening manager and incidental pulmonary findings manager, pulmonary nodule clinic or multidisciplinary team orchestrator.

17. The method of claim 1, wherein the cloud computing service further comprises one or more additional applications that the analytics application can process in combinations.

18. A non-transitory, computer readable storage medium comprising instructions that, when executed by one or more hardware processors, cause the one or more hardware processors to perform the method of claim 1.

19. The non-transitory, computer readable storage medium of claim 18, wherein the ETL pipelines comprise NiFi ETL pipelines.

20. A computing device configured to perform the method of claim 1, the computing device comprising:
one or more hardware processors; and
memory comprising a lung nodule management application and a lung analytics application used in conjunction with the lung nodule management application, the lung analytics application executable by the one or more hardware processors, the lung analytics application comprising:
an entity tree;
NiFi ETL pipelines configured to selectively process workflows and events responsive to trigger points in the workflows;
an analytics data structure configured with plural data structures for monitoring lung screening events, lung screening diagnostic follow-up events, lung incidental events, and lung incidental diagnostic follow-up events; and
one or more analytic dashboards configured to render visualizations of the data stored in the plural data structures of the analytics data structure and derived metrics or key performance indicators.
